(19) United States
(12) Patent Application Publication
(10) Pub. No.: US 2021/0371869 A1
(43) Pub. Date: Dec. 2, 2021

(54) TRANSCRIPTION FACTOR NtERF241 AND METHODS OF USING THE SAME

(71) Applicant: 22nd Century Limited, LLC, Williamsville, NY (US)

(72) Inventor: Paul Rushton, Buffalo, NY (US)

(73) Assignee: 22nd Century Limited, LLC, Williamsville, NY (US)

(21) Appl. No.: 17/138,688

(22) Filed: Dec. 30, 2020

Related U.S. Application Data
(63) Continuation of application No. 16/329,755, filed on Feb. 28, 2019, now abandoned.
(60) Provisional application No. 62/382,895, filed on Sep. 2, 2016.

(51) Int. Cl. C12N 15/82; C07K 14/415; C12N 15/113

(52) U.S. Cl. CPC C12N 15/8213; C12N 15/113; C12N 15/8243; C07K 14/415

(57) ABSTRACT

The present technology provides transcription factors for modifying plant metabolism and nucleic acid molecules that encode such transcription factors. Also provided are methods of using these nucleic acids to modulate alkaloid production in plants and for producing plants and cells having altered alkaloid content.

### CROSS-REFERENCE TO RELATED APPLICATION

This application is a continuation of U.S. patent application Ser. No. 16/329,755, filed Feb. 28, 2019, which is the U.S. National Stage of International Patent Application No. PCT/US2017/049555, filed Aug. 31, 2017, which claims priority from U.S. Provisional Patent Application No. 62/382,895, filed on Sep. 2, 2016, the contents of these applications are hereby incorporated by reference in their entirety.

### TECHNICAL FIELD

The present technology relates generally to transcription factors for modifying plant metabolism, nucleic acid molecules that encode such transcription factors, and methods of using these nucleic acids to modulate alkaloid production in plants and for producing plants and plant cells having altered alkaloid content.

### BACKGROUND

The following description is provided to assist the understanding of the reader. None of the information provided or references cited is admitted to be prior art.

Plant natural products have long been used to enhance human health and social life. Among such bioactive natural plant products are alkaloids, which comprise a class of nitrogen-containing secondary metabolites. Examples of alkaloids include morphine, scopolamine, camptothecin, cocaine, and nicotine. Nicotine, a pyrrolidine alkaloid, is among the most abundant alkaloids produced in Nicotiana spp., and is synthesized in the roots and then translocates through the plant vascular system to the leaves and other aerial tissues where it serves as a defensive compound against herbivores. Nicotine production from a precursor, polyamine putrescine, can be accomplished via two pathways in plants. Putrescine can be synthesized directly from either ornithine or arginine via the activity of decarboxylating enzymes, ornithine decarboxylase (ODC) or arginine decarboxylase (ADC), respectively. The first committed step in nicotine biosynthesis is the conversion of putrescine to N-methylputrescine by putrescine N-methyltransferase (PMT). N-methylputrescine is subsequently oxidized by a diamine oxidase (DAO), and is cyclized to produce a 1-methyl-Δ¹-pyrrolium cation, which is subsequently condensed with nicotinic acid to produce nicotine.

The regulation of gene expression at the level of transcription is a major point of control in many biological processes, including plant metabolism and nicotine biosynthesis. Accordingly, there is a need to identify additional modulators of the nicotine biosynthetic pathway and for compositions and improved methods for genetically regulating the production levels of nicotine and other alkaloids in plants, including transgenic plants, transgenic tobacco plants, recombinant stable cell lines, recombinant stable tobacco cell lines, and derivatives thereof.

### SUMMARY

Disclosed herein are methods and compositions for modulating alkaloid biosynthesis in plants.

In one aspect, the present disclosure provides an isolated cDNA molecule comprising a nucleotide sequence selected from the group consisting of: (a) a nucleotide sequence set forth in SEQ ID NO: 2; (b) a nucleotide sequence that encodes a polypeptide having the amino acid sequence set forth in SEQ ID NO: 3; and (c) a nucleotide sequence that is at least about 90% identical to the nucleotide sequences of (a) or (b), and which encodes a transcription factor that positively regulates nicotinic alkaloid biosynthesis, wherein the nucleotide sequence is operably linked to a heterologous nucleic acid.

In another aspect, the present disclosure provides an expression vector comprising an isolated cDNA molecule comprising a nucleotide sequence selected from the group consisting of: (a) a nucleotide sequence set forth in SEQ ID NO: 2; (b) a nucleotide sequence that encodes a polypeptide having the amino acid sequence set forth in SEQ ID NO: 3; and (c) a nucleotide sequence that is at least about 90% identical to the nucleotide sequences of (a) or (b), and which encodes a transcription factor that positively regulates nicotinic alkaloid biosynthesis, wherein the nucleotide sequence is operably linked to one or more control sequences suitable for directing expression in a Nicotiana host cell.

In another aspect, the present disclosure provides a genetically engineered nicotinic alkaloid-producing Nicotiana plant comprising a cell comprising a chimeric nucleic acid construct comprising an isolated cDNA molecule comprising a nucleotide sequence selected from the group consisting of: (a) a nucleotide sequence set forth in SEQ ID NO: 2; (b) a nucleotide sequence that encodes a polypeptide having the amino acid sequence set forth in SEQ ID NO: 3; and (c) a nucleotide sequence that is at least about 90% identical to the nucleotide sequences of (a) or (b), and which encodes a transcription factor that positively regulates nicotinic alkaloid biosynthesis, wherein the nucleotide sequence is operably linked to a heterologous nucleic acid.

In some embodiments, the engineered Nicotiana plant is a Nicotiana tabacum plant.

In some embodiments, the present disclosure provides seeds from the genetically engineered Nicotiana plant, wherein the seeds comprise the chimeric nucleic acid construct.

In some embodiments, the present disclosure provides a tobacco product comprising the genetically engineered Nicotiana plant or portions thereof.

In some embodiments, the isolated cDNA molecule comprises the nucleotide sequence set forth in SEQ ID NO: 2.

In some embodiments, the isolated cDNA molecule comprises a nucleotide sequence encoding a polypeptide having the amino acid sequence set forth in SEQ ID NO: 3.

In some embodiments, the isolated cDNA molecule comprises a nucleotide sequence that is at least about 90% identical to the nucleotide sequence of SEQ ID NO: 2, and which encodes a transcription factor that positively regulates nicotinic alkaloid biosynthesis.

In some embodiments, the isolated cDNA molecule comprises a nucleotide sequence that is at least about 90% identical to a nucleotide sequence encoding a polypeptide having the amino acid sequence set forth in SEQ ID NO: 3, and which encodes a transcription factor that positively regulates nicotinic alkaloid biosynthesis.

In one aspect, the present disclosure provides a method for increasing a nicotinic alkaloid in a Nicotiana plant, comprising: (a) introducing into a Nicotiana plant an expression vector comprising a nucleotide sequence selected from the group consisting of: (i) a nucleotide sequence set forth in SEQ ID NO: 2; (ii) a nucleotide sequence that encodes a polypeptide having the amino acid sequence set forth in SEQ ID NO: 3; and (iii) a nucleotide sequence that is at least about 90% identical to the nucleotide sequences of (i) or (ii), and which encodes a transcription factor that positively regulates nicotinic alkaloid biosynthesis; and (b) growing the plant under conditions which allow for the expression of a transcription factor that positively regulates nicotinic alkaloid biosynthesis from the nucleotide sequence; wherein expression of the transcription factor results in the plant having an increased nicotinic alkaloid content as compared to a control plant grown under similar conditions.

In some embodiments, the method further comprises overexpressing within the Nicotiana plant at least one of NBB1, A622, quinolate phosphoribosyltransferase (QPT), putrescine N-methyltransferase (PMT), or N-methylputrescine oxidase (MPO).

In some embodiments, the method further comprises overexpressing within the Nicotiana plant at least one additional transcription factor that positively regulates nicotinic alkaloid biosynthesis. In some embodiments, the additional transcription factor that positively regulates nicotinic alkaloid biosynthesis is at least one of NtMYC1a, NtMYC1b, NtMYC2a, or NtMYC2b.

In some embodiments of the method, the expression vector comprises the nucleotide sequence set forth in SEQ ID NO: 2.

In some embodiments of the method, the expression vector comprises a nucleotide sequence that encodes a polypeptide having the amino acid sequence set forth in SEQ ID NO: 3.

In some embodiments of the method, the expression vector comprises a nucleotide sequence that is (a) at least about 90% identical to (i) the nucleotide sequence set forth in SEQ ID NO: 2; or (ii) a nucleotide sequence that encodes a polypeptide having the amino acid sequence set forth in SEQ ID NO: 3, and (b) which encodes a transcription factor that positively regulates nicotinic alkaloid biosynthesis.

In some embodiments of the method, a genetically-engineered Nicotiana plant is provided, wherein the plant has increased expression of a transcription factor that positively regulates nicotinic alkaloid biosynthesis and increased alkaloid content as compared to a control plant.

In some embodiments of the method, a product comprising the engineered plant or portions thereof is provided, wherein the product has an increased nicotinic alkaloid content as compared to a product produced from a control plant. In some embodiments of the method, seeds from the genetically-engineered plant are provided.

In one aspect, the present disclosure provides a method for reducing a nicotinic alkaloid in a Nicotiana plant, comprising down-regulating a transcription factor that positively regulates alkaloid biosynthesis, wherein the transcription factor is down-regulated by: (a) introducing into a Nicotiana plant cell a nucleic acid comprising at least about 15 consecutive nucleotides of a cDNA molecule comprising a nucleotide sequence selected from the group consisting of: (i) a nucleotide sequence set forth in SEQ ID NO: 2; (ii) a nucleotide sequence that encodes a polypeptide having the amino acid sequence set forth in SEQ ID NO: 3; and (iii) a nucleotide sequence that is at least about 90% identical to the nucleotide sequences of (i) or (ii), and which encodes a transcription factor that positively regulates alkaloid biosynthesis; wherein the consecutive nucleotides are in sense orientation, antisense orientation, or both; (b) producing a plant comprising the plant cell; and (c) growing the plant under conditions whereby the nucleotide sequence decreases levels of the transcription factor in the plant as compared to a control plant grown under similar conditions.

In some embodiments, the method further comprises suppressing within the plant at least one of NBB1, A622, quinolate phosphoribosyltransferase (QPT), putrescine-N-methyltransferase (PMT), or N-methylputrescine oxidase (MPO).

In one aspect, the present disclosure provides a method for reducing a nicotinic alkaloid in a Nicotiana plant, comprising down-regulating a transcription factor that positively regulates alkaloid biosynthesis, wherein the transcription factor is down-regulated by: (a) introducing into a population of plant cells a reagent for site-directed mutagenesis of a target comprising at least about 15 consecutive nucleotides of a cDNA molecule comprising a nucleotide sequence selected from the group consisting of: (i) a nucleotide sequence set forth in SEQ ID NO: 2; (ii) a nucleotide sequence that encodes a polypeptide having the amino acid sequence set forth in SEQ ID NO: 3; and (iii) a nucleotide sequence that is at least about 90% identical to the nucleotide sequences of (i) or (ii), and which encodes a transcription factor that positively regulates alkaloid biosynthesis; and (b) detecting and selecting a target mutated plant cell or a plant derived from such a cell, wherein the target mutated plant cell or plant has a mutation in a gene encoding transcription factor positively regulating alkaloid biosynthesis and reduced alkaloid content as compared to a control plant.

In some embodiments of the method, the reagent is a recombinagenic oligonucleobase. In some embodiments, the reagent is a targeted nuclease.

In some embodiments of the method, a mutated plant is provided, wherein the plant has reduced expression of a transcription factor that positively regulates nicotinic alkaloid biosynthesis and reduced alkaloid content as compared to a control plant.

In some embodiments of the method, a product comprising the mutated plant or portions thereof is provided, wherein the product has a reduced level of a nicotinic alkaloid as compared to a product produced from a control plant. In some embodiments of the method, seeds from the mutated plant are provided.

In one aspect, the present disclosure provides a method for reducing nicotinic alkaloid levels in a population of Nicotiana plants, comprising: (a) providing a population of mutated Nicotiana plants; (b) detecting and selecting a target mutated plant within the population, wherein (i) the target mutated plant has decreased expression of a transcription factor that positively regulates alkaloid biosynthesis as compared to a control plant, (ii) the detection comprises using a cDNA molecule as a primer or a probe, and (iii) the cDNA molecule comprises a nucleotide sequence selected from the group consisting of: (1) a nucleotide sequence set forth in SEQ ID NO: 2; (2) a nucleotide sequence that encodes a polypeptide having the amino acid sequence set forth in SEQ ID NO: 3; and (3) a nucleotide sequence that is at least about 90% identical to the nucleotide sequences of (1) or (2), and which encodes a transcription factor that positively regulates alkaloid biosynthesis; and (c) selectively breeding the target mutated plant to produce a population of plants having decreased expression of a transcription factor that positively regulates alkaloid biosynthesis as compared to a population of control plants.

In some embodiments of the method, a mutated alkaloid-producing Nicotiana plant is provided, wherein the plant has reduced expression of a transcription factor that positively regulates alkaloid biosynthesis and reduced alkaloid content, as compared to a control plant.

In some embodiments, the mutated plant is a Nicotiana tabacum plant.

In some embodiments of the method, a tobacco product comprising the mutated plant or portions thereof is provided, wherein the product has a reduced level of a nicotinic alkaloid as compared to a product produced from a control plant. In some embodiments of the method, seeds from the mutated plant are provided.

In one aspect, the present disclosure provides a genetically engineered tobacco plant overexpressing a gene product encoded by SEQ ID NO: 2, wherein the genetically engineered plant exhibits increased expression of the gene product as compared to a control and the genetically engineered plant comprises cells comprising a nucleic acid construct comprising in the 5′ to 3′ direction: (a) a promoter operable in the plant cell, and (b) a heterologous nucleotide sequence operably associated with the promoter, wherein the heterologous nucleotide sequence comprises the nucleotide sequence set forth in SEQ ID NO: 2.

In some embodiments, progeny of the genetically engineered plant are provided, wherein the progeny have overexpression of a gene product encoded by SEQ ID NO: 2.

In one aspect, the present disclosure provides a method of making a genetically engineered increased-nicotine tobacco cell having overexpression of a gene product encoded by SEQ ID NO: 2, the method comprising introducing into the cell an isolated cDNA molecule comprising a nucleotide sequence selected from the group consisting of: (a) a nucleotide sequence set forth in SEQ ID NO: 2; (b) a nucleotide sequence that encodes a polypeptide having the amino acid sequence set forth in SEQ ID NO: 3; and (c) a nucleotide sequence that is at least about 90% identical to the nucleotide sequences of (a) or (b), and which encodes a transcription factor that positively regulates nicotinic alkaloid biosynthesis, wherein the nucleotide sequence is operably linked to a heterologous nucleic acid, to genetically engineer overexpression of a gene product encoded by SEQ ID NO 2. In some embodiments, a tobacco plant cell produced by the method is provided.

In some embodiments, the method further comprises genetically engineering overexpression within the tobacco cell of at least one additional transcription factor that positively regulates nicotinic alkaloid biosynthesis. In some embodiments, the additional transcription factor that positively regulates nicotinic alkaloid biosynthesis is at least one of NtMYC1a, NtMYC1b, NtMYC2a, or NtMYC2b.

In some embodiments, the method further comprises genetically engineering overexpression within the tobacco cell of one or more nicotinic alkaloid biosynthesis enzymes selected from the group consisting of NBB1, A622, quinolate phosphoribosyltransferase (QPT), putrescine-N-methyltransferase (PMT), or N-methylputrescine oxidase (MPO).

The inventions described and claimed herein have many attributes and embodiments including, but not limited to, those set forth or described or referenced in this brief summary. It is not intended to be all-inclusive and the inventions described and claimed herein are not limited to or by the features or embodiments identified in this brief summary, which is included for purposes of illustration only and not restriction. Additional embodiments may be disclosed in the detailed description below.

### DETAILED DESCRIPTION

### I. Introduction

The present technology relates to the discovery of a gene, NtERF241, that is predicted to encode a transcription factor that regulates the nicotinic alkaloid biosynthetic pathway. The nucleic acid sequence of the gene has been determined. The full-length sequence of NtERF241, including the coding region and its 5′ and 3′ upstream and downstream regulatory sequences, is set forth in SEQ ID NO: 1. The open reading frame (ORF) of SEQ ID NO: 1, set forth in SEQ ID NO: 2, is predicted to encode the polypeptide sequence set forth in SEQ ID NO: 3.

Ethylene-responsive element binding factors (ERFs) are members of a family of transcription factors that are specific to plants. A highly conserved DNA binding domain known as the ERF domain is a unique feature of this protein family. Several known ERFs, display GCC box-specific binding activity and have been shown to regulate transcription in plants. For example, ERF transcription factors, including NtERF1, NtERF32, and NtERF121, have been shown to specifically bind the GCC box-like element of the GAG motif required for methyl jasmonate (MeJA)-induced transcription of NtPMT1a, one of the N. tabacum genes encoding putrescine N-methyltransferase (PMT), the first committed step in the synthesis of the nicotine pyrrolidine ring. Sears et al., Plant Mol. Biol., 84:49-66 (2014). The GAG motif of PMT promoters confers the recruitment of ERF and Myc transcription factors. In vitro and in vivo studies have shown that NtERF32 functions as a transcriptional activator of NtPMT genes. Overexpression of NtERF32 has been shown to increase in vivo expression of NtPMT1a and total alkaloid content, while RNAi-mediated knockdown of NtERF32 reduces mRNA levels of several genes in the nicotine biosynthetic pathway, including NtPMT1a and quinolinate phosphoribosyltransferase (NtQPT2), and lowers nicotine and total alkaloid levels. Sears et al. (2014). At the DNA level, NtERF32 and a previously unknown gene, NtERF241, are approximately 90% identical. Accordingly, NtERF241 is predicted to encode an ERF transcription factor that positively regulates genes involved in the biosynthesis of tobacco alkaloids.

Thus, in some embodiments, the present technology provides a previously undiscovered gene (NtERF241) or biologically active fragments thereof that may be used to genetically manipulate the synthesis of alkaloids (e.g., nicotinic alkaloids) in plants that naturally produce alkaloids. For example, Nicotiana spp. (e.g., N. tabacum, N. rustica, and N. benthamiana) naturally produce nicotinic alkaloids. N. tabacum is an agricultural crop and biotechnological uses of this plant continue to increase. The NtERF241 gene or biologically active fragments thereof may be used in plants or plant cells to increase synthesis of nicotinic alkaloids and related compounds, which may have therapeutic applications. In some embodiments, the present technology provides methods for increasing nicotine alkaloid production in plants and plant cells by genetically engineering overexpression of NtERF241. In some embodiments, the present technology provides methods for increasing nicotine alkaloid production in plants and plant cells by genetically engineering overexpression of NtERF241 and at least one MYC transcription factor gene selected from the group consisting of NtMYC1a, NtMYC1b, NtMYC2a, and NtMYC2b. The open reading frame (ORF) of the NtMYC1a gene, set forth in SEQ ID NO: 4, encodes the polypeptide sequence set forth in SEQ ID NO: 5. The ORF of the NtMYC1b gene, set forth in SEQ ID NO: 6, encodes the polypeptide sequence set forth in SEQ ID NO: 7. The full-length sequence of the NtMYC2a gene is set forth in SEQ ID NO: 8. The NtMYC2a polypeptide sequence is set forth in SEQ ID NO: 9. The full-length sequence of the NtMYC2b gene is set forth in SEQ ID NO: 10. The NtMYC2b polypeptide sequence is set forth in SEQ ID NO: 11. In some embodiments, a synergistic effect on the production of nicotinic alkaloids is produced by the combined overexpression of NtERF241 and at least one MYC transcription factor gene selected from the group consisting of NtMYC1a, NtMYC1b, NtMYC2a, and NtMYC2b. NtERF241 or biologically active fragments thereof may also be used to genetically engineer suppression of nicotinic alkaloid synthesis to create tobacco varieties containing zero or low nicotine levels for use as low-toxicity production platforms for the production of plant-made pharmaceuticals (e.g., recombinant proteins and antibodies) or as industrial, food, and biomass crops.

### II. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

An “alkaloid” is a nitrogen-containing basic compound found in plants and produced by secondary metabolism. A “pyrrolidine alkaloid” is an alkaloid containing a pyrrolidine ring as part of its molecular structure, for example, nicotine. Nicotine and related alkaloids are also referred to as pyridine alkaloids in the published literature. A “pyridine alkaloid” is an alkaloid containing a pyridine ring as part of its molecular structure, for example, nicotine. A “nicotinic alkaloid” is nicotine or an alkaloid that is structurally related to nicotine and that is synthesized from a compound produced in the nicotine biosynthesis pathway. Illustrative nicotinic alkaloids include but are not limited to nicotine, nornicotine, anatabine, anabasine, anatalline, N-methylanatabine, N-methylanabasine, myosmine, anabaseine, formylnornicotine, nicotyrine, and cotinine. Other very minor nicotinic alkaloids in tobacco leaf are reported, for example, in Hecht et al., Accounts of Chemical Research 12: 92-98 (1979); Tso, T. G., Production, Physiology and Biochemistry of Tobacco Plant. Ideals Inc., Beltsville, Mo. (1990).

As used herein “alkaloid content” means the total amount of alkaloids found in a plant, for example, in terms of pg/g dry weight (DW) or ng/mg fresh weight (FW). “Nicotine content” means the total amount of nicotine found in a plant, for example, in terms of mg/g DW or FW.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant (e.g., “NtERF241 overexpression”).

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. A plant is genetically engineered when a polynucleotide sequence is introduced that results in the expression of a novel gene in the plant, or an increase in the level of a gene product that is naturally found in the plants. In the present context, “genetically engineered” includes transgenic plants and plant cells, as well as plants and plant cells produced by means of targeted mutagenesis effected, for example, through the use of chimeric RNA/DNA oligonucleotides, as described by Beetham et al., Proc. Natl. Acad. Sci. U.S.A. 96: 8774-8778 (1999) and Zhu et al., Proc. Natl. Acad Sci. U.S;A. 96: 8768-8773 (1999), or so-called “recombinagenic olionucleobases,” as described in International patent publication WO 2003/013226. Likewise, a genetically engineered plant or plant cell may be produced by the introduction of a modified virus, which, in turn, causes a genetic modification in the host, with results similar to those produced in a transgenic plant. See, e.g., U.S. Pat. No. 4,407,956. Additionally, a genetically engineered plant or plant cell may be the product of any native approach (i.e., involving no foreign nucleotide sequences), implemented by introducing only nucleic acid sequences derived from the host plant species or from a sexually compatible plant species. See, e.g., U.S. Patent Application No. 2004/0107455.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Decreased alkaloid plant” or “reduced alkaloid plant” encompasses a genetically engineered plant that has a decrease in alkaloid content to a level less than 50%, and preferably less than 10%, 5%, or 1% of the alkaloid content of a control plant of the same species or variety.

“Increased alkaloid plant” encompasses a genetically engineered plant that has an increase in alkaloid content greater than 10%, and preferably greater than 50%, 100%, or 200% of the alkaloid content of a control plant of the same species or variety.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant (e.g., “NtERF241 down-regulation”).

As used herein, a “synergistic effect” refers to a greater-than-additive effect which is produced by a combination of at least two compounds (e.g., the effect produced by a combined overexpression of at least two transcription factors, such as NtERF241 and at least one one MYC transcription factor gene preferably selected from the group consisting of NtMYC1a, NtMYC1b, NtMYC2a, and NtMYC2b), and which exceeds that which would otherwise result from the individual compound (e.g., the effect produced by the overexpression of a single transcription factor, such as NtERF241 alone).

“Tobacco” or “tobacco plant” refers to any species in the Nicotiana genus that produces nicotinic alkaloids, including but not limited to the following: Nicotiana acaulis, Nicotiana acuminata, Nicotiana acuminata var. multzjlora, Nicotiana africana, Nicotiana alata, Nicotiana amplexicaulis, Nicotiana arentsii, Nicotiana attenuata, Nicotiana benavidesii, Nicotiana benthamiana, Nicotiana bigelovii, Nicotiana bonariensis, Nicotiana cavicola, Nicotiana clevelandii, Nicotiana cordifolia, Nicotiana corymbosa, Nicotiana debneyi, Nicotiana excelsior, Nicotiana forgetiana, Nicotiana fragrans, Nicotiana glauca, Nicotiana glutinosa, Nicotiana goodspeedii, Nicotiana gossei, Nicotiana hybrid, Nicotiana ingulba, Nicotiana kawakamii, Nicotiana knightiana, Nicotiana langsdorfi, Nicotiana linearis, Nicotiana longiflora, Nicotiana maritima, Nicotiana megalosiphon, Nicotiana miersii, Nicotiana noctiflora, Nicotiana nudicaulis, Nicotiana obtusifolia, Nicotiana occidentalis, Nicotiana occidentalis subsp. hesperis, Nicotiana otophora, Nicotiana paniculata, Nicotiana pauczjlora, Nicotiana petunioides, Nicotiana plumbaginifolia, Nicotiana quadrivalvis, Nicotiana raimondii, Nicotiana repanda, Nicotiana rosulata, Nicotiana rosulata subsp. ingulba, Nicotiana rotundifolia, Nicotiana rustica, Nicotiana setchellii, Nicotiana simulans, Nicotiana solanifolia, Nicotiana spegauinii, Nicotiana stocktonii, Nicotiana suaveolens, Nicotiana sylvestris, Nicotiana tabacum, Nicotiana thyrsiflora, Nicotiana tomentosa, Nicotiana tomentosifomis, Nicotiana trigonophylla, Nicotiana umbratica, Nicotiana undulata, Nicotiana velutina, Nicotiana wigandioides, and interspecific hybrids of the above.

“Tobacco product” refers to a product comprising material produced by a Nicotiana plant, including for example, cut tobacco, shredded tobacco, nicotine gum and patches for smoking cessation, cigarette tobacco including expanded (puffed) and reconstituted tobacco, cigar tobacco, pipe tobacco, cigarettes, cigars, and all forms of smokeless tobacco such as chewing tobacco, snuff, snus, and lozenges.

A “transcription factor” is a protein that binds that binds to DNA regions, typically promoter regions, using DNA binding domains and increases or decreases the transcription of specific genes. A transcription factor “positively regulates” alkaloid biosynthesis if expression of the transcription factor increases the transcription of one or more genes encoding alkaloid biosynthesis enzymes and increases alkaloid production. A transcription factor “negatively regulates” alkaloid biosynthesis if expression of the transcription factor decreases the transcription of one or more genes encoding alkaloid biosynthesis enzymes and decreases alkaloid production. Transcription factors are classified based on the similarity of their DNA binding domains. (See, e.g., Stegmaier et al., Genome Inform. 15 (2): 276-86 ((2004)). Classes of plant transcription factors include ERF transcription factors; Myc basic helix-loop-helix transcription factors; homeodomain leucine zipper transcription factors; AP2 ethylene-response factor transcription factors; and B3 domain, auxin response factor transcription factors.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “biologically active fragment” means a fragment of NtERF241 which can, for example, bind to an antibody that will also bind the full length NtERF241. The term “biologically active fragment” can also mean a fragment of NtERF241 which can, for example, be useful in induction of gene silencing in plants. In some embodiments, a biologically active fragment of NtERF241 can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the full length sequence (either amino acid or nucleic acid). SEQ ID NO. 3, which depicts the full length amino acid sequence of NtERF241, is 246 amino acids. In other embodiments, a biologically active peptide fragment of NtERF241 can be, for example, at least about 5 contiguous amino acids. In yet other embodiments, the biologically active peptide fragment of NtERF241 can be about 5 contiguous amino acids up to about 245 contiguous amino acids, or any value of contiguous amino acids in between these two amounts, such as but not limited to about 7, about 8, about 9, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 245 contiguous amino acids. SEQ ID NO. 2 depicts the ORF of SEQ ID NO: 1, which depicts the full-length sequence of NtERF241, including the coding region and its 5′ and 3′ upstream and downstream regulatory sequences. SEQ ID NO. 2 is 741 base pairs in length. In some embodiments, a biologically active nucleic acid fragment of NtERF241 can be, for example, at least about 15 contiguous nucleic acids. In yet other embodiments, the biologically active nucleic acid fragment of NtERF241 can be about 15 contiguous nucleic acids up to about 740 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, or about 740 contiguous nucleic acids.

### III. Modulating Alkaloid Production in Plants

The disclosure of the present technology relates to the use of NtERF241 or biologically active fragments thereof in compositions and methods for modulating alkaloid production in plants.

A. Increasing Alkaloid Production

In some embodiments, the present technology relates to increasing alkaloids in plants by overexpressing a transcription factor with a positive regulatory effect on alkaloid production. The NtERF241 gene or its open reading frame may be used to engineer overproduction of alkaloids, for example, nicotinic alkaloids (e.g., nicotine) in plants or plant cells.

Alkaloids, such as nicotine, can be increased by overexpressing one or more genes encoding enzymes in the alkaloid biosynthesis pathway. See, e.g., Sato et al., Proc. Natl. Acad. Sci. U.S.A. 98(1):367-72 (2001). The effect of overexpressing PMT alone on nicotine content of leaves yields an increase of only 40%, despite 4- to 8-fold increases in PMT transcript levels in roots, suggesting that limitations at other steps of the pathway prevented a larger effect. Accordingly, the present technology contemplates that overexpressing a transcription factor with a predicted positive regulatory effect on alkaloid production (e.g., NtERF241) and at least one at least one alkaloid biosynthesis gene, such as A622, NBB1, QPT, PMT, and/or MPO, will result in greater alkaloid production than up-regulating the alkaloid biosynthesis gene alone.

Pursuant to this aspect of the present technology, a nucleic acid construct comprising NtERF241, its open reading frame, or a biologically active fragment thereof, and at least one of A622, NBB1, QPT, PMT, or MPO is introduced into a plant cell. An illustrative nucleic acid construct may comprise, for example, both NtERF241 or a biologically active fragment thereof and QPT. Similarly, for example, a genetically engineered plant overexpressing NtERF241 and QPT may be produced by crossing a transgenic plant overexpressing NtERF241 with a transgenic plant overexpressing QPT. Following successive rounds of crossing and selection, a genetically engineered plant overexpressing NtERF241 and QPT can be selected.

B. Decreasing Alkaloid Production

Alkaloid production may be reduced by suppression of an endogenous gene encoding a transcription factor that positively regulates alkaloid production using the NtERF241 transcription factor gene sequence of the present technology in a number of ways generally known in the art, for example, RNA interference (RNAi) techniques, artificial microRNA techniques, virus-induced gene silencing (VIGS) techniques, antisense techniques, sense co-suppression techniques, and targeted mutagenesis techniques. Accordingly, the present technology provides methodology and constructs for decreasing alkaloid content in a plant by suppressing NtERF241. Suppressing more than one gene encoding a transcription factor that positively regulates alkaloid production (e.g., NtMYC1a, NtMYC1b, NtMYC2a, and/or NtMYC2b) may further decrease alkaloids levels in a plant.

Previous reports indicate that suppressing an alkaloid biosynthesis gene in Nicotiana decreases nicotinic alkaloid content. For example, suppressing QPT reduces nicotine levels. (See, e.g., U.S. Pat. No. 6,586,661). Suppressing A622 or NBB1 also reduces nicotine levels (see, e.g., WO 2006/109197), as does suppressing PMT (see, e.g., Chintapakorn & Hamill, Plant Mol. Biol. 53:87-105 (2003)) or MPO (see, e.g., WO 2008/020333 and WO 2008/008844; Katoh et al., Plant Cell Physiol. 48(3): 550-4 (2007)). Accordingly, the present technology contemplates further decreasing nicotinic alkaloid content by suppressing one or more of A622, NBB1, QPT, PMT, and MPO, and suppressing NtERF241. Pursuant to this aspect of the present technology, a nucleic acid construct comprising at least a biologically active fragment of NtERF241 and at least a biologically active fragment of one or more of A622, NBB1, QPT, PMT, and MPO are introduced into a cell or plant. An illustrative nucleic acid construct may comprise both a biologically active fragment of NtERF241 and QPT.

C. Genetic Engineering of Plants and Cells Using Transcription Factor Sequences that Regulate Alkaloid Production

Transcription Factor Sequences

Transcription factor genes of the present technology include the sequences set forth in SEQ ID NO: 1 and SEQ ID NO: 2, including biologically active fragments thereof of at least about 15 contiguous nucleic acids up to about 740 contiguous nucleic acids, or any value of contiguous nucleic acids in between these two amounts, such as but not limited to about 20, about 30, about 40, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, or about 740 contiguous nucleic acids. In some embodiments, transcription factor genes of the present technology include the sequences set forth in SEQ ID NO: 1 and SEQ ID NO: 2, including biologically active fragments thereof of at least about 21 consecutive nucleotides, which are of a sufficient length as to be useful in induction of gene silencing in plants (Hamilton & Baulcombe, Science, 286:950-952 (1999)).

The present technology also includes “variants” of SEQ ID NO: 1 and SEQ ID NO: 2, with one or more bases deleted, substituted, inserted, or added, which variant codes for a polypeptide that regulates alkaloid biosynthesis activity. Accordingly, sequences having “base sequences with one or more bases deleted, substituted, inserted, or added” retain physiological activity even when the encoded amino acid sequence has one or more amino acids substituted, deleted, inserted, or added. Additionally, multiple forms of NtERF241 may exist, which may be due to post-translational modification of a gene product, or to multiple forms of the transcription factor gene. Nucleotide sequences that have such modifications and that code for an NtERF241 transcription factor that regulates alkaloid biosynthesis are included within the scope of the present technology.

For example, the poly A tail or 5′- or 3′-end, nontranslated regions may be deleted, and bases may be deleted to the extent that amino acids are deleted. Bases may also be substituted, as long as no frame shift results. Bases also may be “added” to the extent that amino acids are added. However, it is essential that any such modification does not result in the loss of transcription factor activity that regulates alkaloid biosynthesis. A modified DNA in this context can be obtained by modifying the DNA base sequences of the present technology so that amino acids at specific sites in the encoded polypeptide are substituted, deleted, inserted, or added by site-specific mutagenesis, for example. (See Zoller & Smith, Nucleic Acid Res. 10:6487-500 (1982)).

A transcription factor sequence can be synthesized ab initio from the appropriate bases, for example, by using an appropriate protein sequence disclosed herein as a guide to create a DNA molecule that, though different from the native DNA sequence, results in the production of a protein with the same or similar amino acid sequence.

Unless otherwise indicated, all nucleotide sequences determined by sequencing a DNA molecule herein were determined using an automated DNA sequencer, such as the Model 3730xl from Applied Biosystems, Inc. Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

For purposes of the present technology, two sequences hybridize under stringent conditions when they form a double-stranded complex in a hybridization solution of 6×SSE, 0.5% SDS, 5×Denhardt's solution and 100 μg of non-specific carrier DNA. See Ausubel, et al., supra, at section 2.9, supplement 27 (1994). Sequences may hybridize at “moderate stringency,” which is defined as a temperature of 60° C. in a hybridization solution of 6×SSE, 0.5% SDS, 5×Denhardt's solution and 100 μg of non-specific carrier DNA. For “high stringency” hybridization, the temperature is increased to 68° C. Following the moderate stringency hybridization reaction, the nucleotides are washed in a solution of 2×SSE plus 0.05% SDS for five times at room temperature, with subsequent washes with 0.1×SSC plus 0.1% SOS at 60° C. for 1 h. For high stringency, the wash temperature is increased to 68° C. For the purpose of the technology, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present technology encompasses nucleic acid molecules which are at least about 90%, about 91%, about 92%, about 93%, about 94%, about 95%, about 96%, about 97%, about 98%, about 99% or 100% identical to a nucleic acid sequence described in any of SEQ ID NOs: 1-2. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

Nucleic Acid Constructs

In some embodiments of the present technology, a sequence that increases the activity of a transcription factor that regulates alkaloid biosynthesis is incorporated into a nucleic acid construct that is suitable for introducing into a plant or cell. Thus, such a nucleic acid construct can be used to overexpress NtERF241, and optionally at least one of A622, NBB1, PMT, QPT, MPO, NtMYC1a, NtMYC1b, NtMYC2a, or NtMYC2b in a plant or cell.

Recombinant nucleic acid constructs may be made using standard techniques. For example, the DNA sequence for transcription may be obtained by treating a vector containing the sequence with restriction enzymes to cut out the appropriate segment. The DNA sequence for transcription may also be generated by annealing and ligating synthetic oligonucleotides or by using synthetic oligonucleotides in a polymerase chain reaction (PCR) to give suitable restriction sites at each end. The DNA sequence then is cloned into a vector containing suitable regulatory elements, such as upstream promoter and downstream terminator sequences.

In some embodiments of the present technology, nucleic acid constructs comprise a sequence encoding a transcription factor (i.e., NtERF241) that regulates alkaloid biosynthesis operably linked to one or more regulatory or control sequences, which drive expression of the transcription factor-encoding sequence in certain cell types, organs, or tissues without unduly affecting normal development or physiology.

Promoters useful for expression of a nucleic acid sequence introduced into a cell to either decrease or increase expression of a transcription factor that regulates alkaloid biosynthesis may be constitutive promoters, such as the carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter). Tissue-specific, tissue-preferred, cell type-specific, and inducible promoters may be desirable under certain circumstances. For example, a tissue-specific promoter allows for overexpression in certain tissues without affecting expression in other tissues.

Exemplary promoters include promoters which are active in root tissues, such as the tobacco RB7promoter (see, e.g., Hsu et al., Pestic. Sci. 44:9-19 (1995); U.S. Pat. No. 5,459,252), maize promoter CRWAQ81 (see, e.g., U.S. Patent Publication No. 2005/0097633); the Arabidopsis ARSK1 promoter (see, e.g., Hwang & Goodman, Plant J. 8:37-43 (1995)), the maize MR7 promoter (see, e.g., U.S. Pat. No. 5,837,848), the maize ZRP2 promoter (see, e.g., U.S. Pat. No. 5,633,363), the maize MTL promoter (see, e.g., U.S. Pat. Nos. 5,466,785 and 6,018,099) the maize MRS1, MRS2, MRS3, and MRS4 promoters (see, e.g., U.S. Patent Publication No. 2005/0010974), an Arabidopsis cryptic promoter (see, e.g., U.S. Patent Publication No. 2003/0106105) and promoters that are activated under conditions that result in elevated expression of enzymes involved in nicotine biosynthesis such as the tobacco RD2 promoter (see, e.g., U.S. Pat. No. 5,837,876), PMT promoters (see, e.g., Shoji et al., Plant Cell Physiol. 41:831-39 (2000); WO 2002/038588), or an A622 promoter (see, e.g., Shoji et al., Plant Mol. Biol. 50:427-40 (2002)).

The vectors of the technology may also contain termination sequences, which are positioned downstream of the nucleic acid molecules of the present technology, such that transcription of mRNA is terminated, and polyA sequences added. Exemplary terminators include Agrobacterium tumefaciens nopaline synthase terminator (Tnos), Agrobacterium tumefaciens mannopine synthase terminator (Tmas), and the CaMV 35S terminator (T35S). Termination regions include the pea ribulose bisphosphate carboxylase small subunit termination region (TrbcS) or the Tnos termination region. The expression vector also may contain enhancers, start codons, splicing signal sequences, and targeting sequences.

Expression vectors of the present technology may also contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

Examples of suitable selectable markers include but are not limited to adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3′-O-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium gluphosinate. See, e.g., Thompson et al., EMBO J. 9:2519-23 (1987)). Other suitable selection markers known in the art may also be used.

Visible markers such as green florescent protein (GFP) may be used. Methods for identifying or selecting transformed plants based on the control of cell division have also been described. See, e.g., WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For example, when Agrobacterium is the host, T-DNA sequences may be included to facilitate the subsequent transfer to and incorporation into plant chromosomes.

Such gene constructs may suitably be screened for activity by transformation into a host plant via Agrobacterium and screening for modified alkaloid levels.

Suitably, the nucleotide sequences for the genes may be extracted from the GenBank™ nucleotide database and searched for restriction enzymes that do not cut. These restriction sites may be added to the genes by conventional methods such as incorporating these sites in PCR primers or by sub-cloning.

Constructs may be comprised within a vector, such as an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors, A Laboratory Manual, Elsevier, Amsterdam (1986). Examples of suitable vectors include the Ti plasmid vectors.

In some embodiments, the present technology provides expression vectors that enable the overexpression of NtERF241, for modulating the production levels of nicotine and other alkaloids, including various flavonoids. In some embodiments, the expression vectors of the present technology further enable the overexpression of at least one of NtMYC1a, NtMYC1b, NtMYC2a, and NtMYC2b. These expression vectors can be transiently introduced into host plant cells or stably integrated into the genomes of host plant cells to generate transgenic plants by various methods known to persons skilled in the art. When these expression vectors are stably integrated into the genomes of host plant cells to generate stable cell lines or transgenic plants, the overexpression of NtERF241 alone or in combination with an alkaloid biosynthesis enzyme or another transcription factor, such as NtMYC1a, NtMYC1b, NtMYC2a, or NtMYC2b, can be deployed as a method for modulating the promoter activation of endogenous promoters that are responsive to this transcription factor. Host plant cells can be further manipulated to receive heterologous promoter constructs that are responsive to NtERF241. Host plant cells can be also be further manipulated to receive heterologous promoter constructs that have been modified by incorporating one or more GAG motifs upstream of the core elements of the heterologous promoter of interest.

Any promoter of interest can be manipulated to be responsive to jasmonic acid (JA) and methyl jasmonate (MeJA) by incorporating one or more GAG motifs and/or derivative GAG motifs upstream of the promoter of interest. Suitable promoters include various promoters of any origin that can be activated by the transcriptional machinery of plant cells, such as various homologous or heterologous plant promoters and various promoters derived from plant pathogens, including bacteria and viruses. Suitable promoters include constitutively active promoters and inducible promoters.

With respect to the expression vectors described below, various genes that encode enzymes involved in biosynthetic pathways for the production of alkaloids, flavonoids, and nicotine can be suitable as transgenes that can be operably linked to a promoter of interest.

In some embodiments, an expression vector comprises a promoter operably linked to the cDNA encoding NtERF241. In another embodiment, a plant cell line comprises an expression vector comprising a promoter operably linked to the cDNA encoding NtERF241. In another embodiment, a transgenic plant comprises an expression vector comprising a promoter operably linked to the cDNA encoding NtERF241. In another embodiment, methods for genetically modulating the production of alkaloids, flavonoids, and nicotine are provided, comprising: introducing an expression vector comprising a promoter operably linked to the cDNA encoding NtERF241. In some embodiments, the expression vector further comprises a promoter operably linked to cDNA encoding at least one of NtMYC1a, NtMYC1b, NtMYC2a, and NtMYC2b.

In another embodiment, an expression vector comprises (i) a first promoter operably linked to cDNA encoding NtERF241, and (ii) a second promoter operably linked to cDNA encoding an enzyme involved in the biosynthesis of alkaloids. In another embodiment, a plant cell line comprises (i) an expression vector comprising a first promoter operably linked to cDNA encoding NtERF241, and (ii) a second promoter operably linked to cDNA encoding an enzyme involved in the biosynthesis of alkaloids. In another embodiment, a transgenic plant comprises (i) an expression vector comprising a first promoter operably linked to cDNA encoding NtERF241, and (ii) a second promoter operably linked to cDNA encoding an enzyme involved in the biosynthesis of alkaloids. In another embodiment, methods for genetically modulating the production level of alkaloids are provided, comprising introducing an expression vector comprising (a) a first promoter operably linked to cDNA encoding NtERF241, and (b) a second promoter operably linked to cDNA encoding an enzyme involved in the biosynthesis of alkaloids. In some embodiments, the expression vector further comprises a promoter operably linked to cDNA encoding at least one of NtMYC1a, NtMYC1b, NtMYC2a, and NtMYC2b. In some embodiments, the enzyme involved in alkaloid biosynthesis comprises one or more of A622, NBB1, quinolate phosphoribosyltransferase (QPT), putrescine N-methyltransferase (PMT), or N-methylputrescine oxidase (MPO).

In another embodiment, an expression vector comprises (i) a first promoter operably linked to cDNA encoding NtERF241, (ii) and a second promoter operably linked to cDNA encoding an enzyme involved in the biosynthesis of flavonoids. In another embodiment, a plant cell line comprises (i) an expression vector comprising a first promoter operably linked to cDNA encoding NtERF241, and (ii) a second promoter operably linked to cDNA encoding an enzyme involved in the biosynthesis of flavonoids. In another embodiment, a transgenic plant comprises an expression vector comprising (i) a first promoter operably linked to cDNA encoding NtERF241, and (ii) a second promoter operably linked to cDNA encoding an enzyme involved in the biosynthesis of flavonoids. In some embodiments, the expression vector further comprises a promoter operably linked to cDNA encoding at least one of NtMYC1a, NtMYC1b, NtMYC2a, and NtMYC2b. In another embodiment, methods for modulating the production level of flavonoids are provided, comprising introducing an expression vector comprising (i) a first promoter operably linked to cDNA encoding NtERF241, and (ii) a second promoter operably linked to cDNA encoding an enzyme involved in the biosynthesis of flavonoids. In some embodiments of the methods, the expression vector further comprises a promoter operably linked to cDNA encoding at least one of NtMYC1a, NtMYC1b, NtMYC2a, and NtMYC2b.

In another embodiment, an expression vector comprises (i) a first promoter operably linked to cDNA encoding NtERF241, and (ii) a second promoter operably linked to cDNA encoding an enzyme involved in nicotine biosynthesis. In another embodiment, a plant cell line comprises an expression vector comprising (i) a first promoter operably linked to cDNA encoding NtERF241, and (ii) a second promoter operably linked to cDNA encoding an enzyme involved in nicotine biosynthesis. In another embodiment, a transgenic plant comprises an expression vector comprising (i) a first promoter operably linked to cDNA encoding NtERF241, and (ii) a second promoter operably linked to cDNA encoding an enzyme involved in nicotine biosynthesis. In some embodiments, the expression vector further comprises a promoter operably linked to cDNA encoding at least one of NtMYC1a, NtMYC1b, NtMYC2a, and NtMYC2b. In some embodiments, the enzyme involved in nicotine biosynthesis is one or more of A622, NBB1, quinolate phosphoribosyltransferase (QPT), putrescine N-methyltransferase (PMT), or N-methylputrescine oxidase (MPO). In some embodiments, the enzyme involved in nicotine biosynthesis is PMT. In another embodiment, methods for genetically modulating the production level of nicotine are provided, comprising introducing an expression vector comprising (i) a first promoter operably linked to cDNA encoding NtERF241, and (ii) a second promoter operably linked to cDNA encoding an enzyme involved in nicotine biosynthesis. In some embodiments of the methods, the expression vector further comprises a promoter operably linked to cDNA encoding at least one of NtMYC1a, NtMYC1b, NtMYC2a, and NtMYC2b.

Another embodiment is directed to an isolated cDNA encoding NtERF241 (SEQ ID NO: 2), or biologically active fragments thereof. Another embodiment is directed to an isolated cDNA encoding NtERF241 and having at least about 90%, about 91%, about 92%, about 93%, about 94%, about 95%, about 96%, about 97%, about 98%, or about 99% sequence identity to SEQ ID NO: 2, or biologically active variant fragments thereof.

Another embodiment is directed to an expression vector comprising a first sequence comprising an isolated cDNA encoding NtERF241 and having at least about 90%, about 91%, about 92%, about 93%, about 94%, about 95%, about 96%, about 97%, about 98%, or about 99% sequence identity to SEQ ID NO: 2, or biologically active fragments thereof. In some embodiments, the expression vector further comprises an additional sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 4, 6, 8, and 10, respectively, or fragments thereof.

Another embodiment is directed to a plant cell line comprising an expression vector comprising an isolated cDNA encoding NtERF241 and having at least about 90%, about 91%, about 92%, about 93%, about 94%, about 95%, about 96%, about 97%, about 98%, or about 99% sequence identity to SEQ ID NO: 2, or fragments thereof. In some embodiments, the expression vector further comprises an additional sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 4, 6, 8, and 10, respectively, or fragments thereof.

Another embodiment is directed to a transgenic plant comprising an expression vector comprising an isolated cDNA encoding NtERF241 and having at least about 90%, about 91%, about 92%, about 93%, about 94%, about 95%, about 96%, about 97%, about 98%, or about 99% sequence identity to SEQ ID NO: 2, or biologically active fragments thereof. In some embodiments, the expression vector further comprises a second sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 4, 6, 8, and 10, respectively, or fragments thereof.

Another embodiment is directed to a method for genetically regulating nicotine levels in plants, comprising introducing into a plant an expression vector comprising an isolated cDNA encoding NtERF241 and having at least about 90%, about 91%, about 92%, about 93%, about 94%, about 95%, about 96%, about 97%, about 98%, or about 99% sequence identity to SEQ ID NO: 2, or fragments thereof. In some embodiments, the expression vector further comprises a second sequence comprising an isolated cDNA encoding at least one of NtMYC1a, NtMYC1b, NtMYC2a, and NtMYC2b, and having at least about 85%, about 90%, about 91%, about 92%, about 93%, about 94%, about 95%, about 96%, about 97%, about 98%, about 99% or 100% sequence identity to SEQ ID NOs: 4, 6, 8, and 10, respectively, or fragments thereof.

Methodology for Suppressing a Transcription Factor that Regulates Alkaloid Production

In some embodiments of the present technology, methods and constructs are provided for suppressing a transcription factor that regulates alkaloid production, altering alkaloid levels, and producing plants with altered alkaloid levels. Examples of methods that may be used for suppressing a transcription factor that regulates alkaloid production (e.g., NtERF241) include antisense, sense co-suppression, RNAi, artificial microRNA, virus-induced gene silencing (VIGS), antisense, sense co-suppression, and targeted mutagenesis approaches.

RNAi techniques involve stable transformation using RNAi plasmid constructs (Helliwell & Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA) techniques exploit the microRNA (miRNA) pathway that functions to silence endogenous genes in plants and other eukaryotes (Schwab et al., Plant Cell 18:1121-33 (2006); Alvarez et al., Plant Cell 18:1134-51 (2006)). In this method, 21-nucleotide-long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes, which share nucleotide identity with the 21 nucleotide amiRNA sequence.

In RNAi silencing techniques, two factors can influence the choice of length of the fragment. The shorter the fragment the less frequently effective silencing will be achieved, but very long hairpins increase the chance of recombination in bacterial host strains. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable, a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences. Many of these same principles apply to selection of target regions for designing amiRNAs.

Virus-induced gene silencing (VIGS) techniques are a variation of RNAi techniques that exploits the endogenous-antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used. Tobacco rattle virus based VIGS systems are described for example, in Baulcombe, Curr. Opin. Plant Biol. 2:109-113 (1999); Lu et al., Methods 30:296-303 (2003); Ratcliff et al., The Plant Journal 25:237-245 (2001); and U.S. Pat. No. 7,229,829.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail in Stam et al., Plant J. 21 27-42 (2000).

Sense co-suppression techniques involve introducing a highly expressed sense transgene into a plant resulting in reduced expression of both the transgene and the endogenous gene (Depicker and van Montagu, Curr. Opin. Cell Biol. 9: 373-82 (1997)). The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant (Henikoff et al., Plant Physiol. 135: 630-6 (2004); Li et al., Plant 27: 235-242 (2001)). TILLING involves treating seeds or individual cells with a mutagen to cause point mutations that are then discovered in genes of interest using a sensitive method for single-nucleotide mutation detection. Detection of desired mutations (e.g., mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e., deletions, in plant genomes that can also be detected using PCR in a manner similar to TILLING.

Host Plants and Cells

In some embodiments, the present technology relates to the genetic manipulation of a plant or cell via introducing a polynucleotide sequence that encodes a transcription factor that regulates alkaloid biosynthesis (e.g., NtERF241). Accordingly, the present technology provides methodology and constructs for reducing or increasing alkaloid synthesis in a plant. Additionally, the present technology provides methods for producing alkaloids and related compounds in a plant cell.

The plants utilized in the present technology may include the class of alkaloid-producing higher plants amenable to genetic engineering techniques, including both monocotyledonous and dicotyledonous plants, as well as gymnosperms. In some embodiments, the alkaloid-producing plant includes a nicotinic alkaloid-producing plant of the Nicotiana, Duboisia, Solanum, Anthocercis, and Salpiglossis genera in the Solanaceae or the Eclipta and Zinnia genera in the Compositae.

As known in the art, there are a number of ways by which genes and gene constructs can be introduced into plants, and a combination of plant transformation and tissue culture techniques have been successfully integrated into effective strategies for creating transgenic crop plants.

These methods, which can be used in the present technology, have been described elsewhere (Potrykus, Annu. Rev. Plant Physiol. Plant Mol. Biol. 42:205-225 (1991); Vasil, Plant Mol. Biol. 5:925-937 (1994); Walden and Wingender, Trends Biotechnol. 13:324-331 (1995); Songstad et al., Plant Cell, Tissue and Organ Culture 40:1-15 (1995)), and are well known to persons skilled in the art. For example, one skilled in the art will certainly be aware that, in addition to Agrobacterium-mediated transformation of Arabidopsis by vacuum infiltration (Bechtold et al., C.R. Acad. Sci. Ser. III Sci. Vie, 316:1194-1199 (1993)) or wound inoculation (Katavic et al., Mol. Gen. Genet. 245:363-370 (1994)), it is equally possible to transform other plant and crop species, using Agrobacterium Ti-plasmid-mediated transformation (e.g., hypocotyl (DeBlock et al., Plant Physiol. 91:694-701 (1989)) or cotyledonary petiole (Moloney et al., Plant Cell Rep. 8:238-242 (1989) wound infection), particle bombardment/biolistic methods (Sanford et al., J. Part. Sci. Technol. 5:27-37 (1987); Nehra et al., Plant J. 5: 285-297 (1994); Becker et al., Plant J. 5: 299-307 (1994)), or polyethylene glycol-assisted protoplast transformation (Rhodes et al., Science 240: 204-207 (1988); Shimamoto et al., Nature 335: 274-276 (1989)) methods.

Agrobacterium rhizogenes may be used to produce transgenic hairy roots cultures of plants, including tobacco, as described, for example, by Guillon et al., Curr. Opin. Plant Biol. 9:341-6 (2006). “Tobacco hairy roots” refers to tobacco roots that have T-DNA from an Ri plasmid of Agrobacterium rhizogenes integrated in the genome and grow in culture without supplementation of auxin and other phytohormones. Tobacco hairy roots produce nicotinic alkaloids as roots of a whole tobacco plant do.

Additionally, plants may be transformed by Rhizobium, Sinorhizobium, or Mesorhizobium transformation. (Broothaerts et al., Nature 433: 629-633 (2005)).

After transformation of the plant cells or plant, those plant cells or plants into which the desired DNA has been incorporated may be selected by such methods as antibiotic resistance, herbicide resistance, tolerance to amino-acid analogues or using phenotypic markers.

Various assays may be used to determine whether the plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

Modified alkaloid content, effected in accordance with the present technology, can be combined with other traits of interest, such as disease resistance, pest resistance, high yield or other traits. For example, a stable genetically engineered transformant that contains a suitable transgene that modifies alkaloid content may be employed to introgress a modified alkaloid content trait into a desirable commercially acceptable genetic background, thereby obtaining a cultivar or variety that combines a modified alkaloid level with said desirable background. For example, a genetically engineered tobacco plant with reduced nicotine may be employed to introgress the reduced nicotine trait into a tobacco cultivar with disease resistance trait, such as resistance to TMV, blank shank, or blue mold. Alternatively, cells of a modified alkaloid content plant of the present technology may be transformed with nucleic acid constructs conferring other traits of interest.

The present technology also contemplates genetically engineering a cell with a nucleic acid sequence encoding a transcription factor that regulates alkaloid biosynthesis (e.g., NtERF241).

Additionally, cells expressing alkaloid biosynthesis genes may be supplied with precursors to increase substrate availability for alkaloid synthesis. Cells may be supplied with analogs of precursors which may be incorporated into analogs of naturally occurring alkaloids.

Constructs according to the present technology may be introduced into any plant cell, using a suitable technique, such as Agrobacterium-mediated transformation, particle bombardment, electroporation, and polyethylene glycol fusion, or cationic lipid-mediated transfection.

Such cells may be genetically engineered with a nucleic acid construct of the present technology without the use of a selectable or visible marker and transgenic organisms may be identified by detecting the presence of the introduced construct. The presence of a protein, polypeptide, or nucleic acid molecule in a particular cell can be measured to determine if, for example, a cell has been successfully transformed or transfected. For example, and as routine in the art, the presence of the introduced construct can be detected by PCR or other suitable methods for detecting a specific nucleic acid or polypeptide sequence. Additionally, genetically engineered cells may be identified by recognizing differences in the growth rate or a morphological feature of a transformed cell compared to the growth rate or a morphological feature of a non-transformed cell that is cultured under similar conditions. See WO 2004/076625.

The present technology also contemplates transgenic plant cell cultures comprising genetically engineered plant cells transformed with the nucleic acid molecules described herein and expressing NtERF241. The cells may also express at least one additional transcription factor gene such as NtMYC1a, NtMYC1b, NtMYC2a, or NtMYC2b, and/or at least one nicotine biosynthesis gene such as A622, NBB1, QPT, PMT, or MPO

The present technology also contemplates cell culture systems comprising genetically engineered cells transformed with the nucleic acid molecules described herein and expressing NtERF241. It has been shown that transgenic hairy root cultures overexpressing PMT provide an effective means for large-scale commercial production of scopolamine, a pharmaceutically important tropane alkaloid. Zhang et al., Proc. Nat'l Acad. Sci. USA 101:6786-91 (2004). Accordingly, large-scale or commercial quantities of nicotinic alkaloids can be produced in tobacco hairy root culture by overexpressing NtERF241. Likewise, the present technology contemplates cell culture systems, such as bacterial or insect cell cultures, for producing large-scale or commercial quantities of nicotinic alkaloids, nicotine analogs, or nicotine precursors by expressing NtERF241. The cells may also express at least one additional transcription factor gene such as NtMYC1a, NtMYC1b, NtMYC2a, or NtMYC2b, and/or at least one nicotine biosynthesis gene such as A622, NBB1, QPT, PMT, or MPO.

D. Quantifying Alkaloid Content

In some embodiments of the present technology, genetically engineered plants and cells are characterized by reduced alkaloid content.

A quantitative reduction in alkaloid levels can be assayed by several methods, as for example by quantification based on gas-liquid chromatography, high performance liquid chromatography, radio-immunoassays, and enzyme-linked immunosorbent assays.

In describing a plant of the present technology, the phrase “decreased alkaloid plant” or “reduced alkaloid plant” encompasses a plant that has a decrease in alkaloid content to a level less than about 50%, about 40%, about 30%, about 25%, about 20%, about 15%, about 10%, about 9%, about 8%, about 7%, about 6%, about 5%, about 4%, about 3%, about 2% or about 1% of the alkaloid content of a control plant of the same species or variety.

In some embodiments of the present technology, genetically engineered plants are characterized by increased alkaloid content. Similarly, genetically engineered cells are characterized by increased alkaloid production.

In describing a plant of the present technology, the phrase “increased alkaloid plant” encompasses a genetically engineered plant that has an increase in alkaloid content greater than about 10%, about 25%, about 30%, about 40%, about 50%, about 75%, about 100%, about 125%, about 150%, about 175%, or about 200% of the alkaloid content of a control plant of the same species or variety.

A successfully genetically engineered cell is characterized by increased alkaloid synthesis. For example, a genetically engineered cell of the present technology may produce more nicotine compared to a control cell.

A quantitative increase in nicotinic alkaloid levels can be assayed by several methods, as for example by quantification based on gas-liquid chromatography, high performance liquid chromatography, radio-immunoassays, and enzyme-linked immunosorbent assays.

### IV. Products

The polynucleotide sequences that encode the NtERF241 transcription factor that is predicted to regulate alkaloid biosynthesis may be used for production of plants with altered alkaloid levels. Such plants may have useful properties, such as increased pest resistance in the case of increased-alkaloid plants, or reduced toxicity and increased palatability in the case of decreased-alkaloid plants.

Plants of the present technology may be useful in the production of products derived from harvested portions of the plants. For example, decreased-alkaloid tobacco plants may be useful in the production of reduced-nicotine cigarettes for smoking cessation. Increased-alkaloid tobacco plants may be useful in the production of modified risk tobacco products.

Additionally, plants and cells of the present technology may be useful in the production of alkaloids or alkaloid analogs including nicotine analogs, which may be used as therapeutics, insecticides, or synthetic intermediates. To this end, large-scale or commercial quantities of alkaloids and related compounds can be produced by a variety of methods, including extracting compounds from genetically engineered plant, cell, or culture system, including but not limited to hairy root cultures, suspension cultures, callus cultures, and shoot cultures.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Transcription Factor NtERF241

The full-length NtERF241 gene (SEQ ID NO: 1) is 1900 bp in length. The open reading frame (ORF) of NtERF241, which is 741 bp in length, is set forth in SEQ ID NO: 2, and is predicted to encode a 246-amino acid polypeptide as set forth in SEQ ID NO: 3. The role that this gene is predicted to play in nicotine biosynthesis has not yet been reported.

The NtERF241 gene was uncovered by searching the SolGenomics database using the nucleic acid and amino acid sequences for NtERF32 (also known as ERF2 or EREBP2). The identified gene, which is not present in the TOBFAC database of ERF tobacco genes, encodes a transcription factor that is similar, but not identical to NtERF32. As the list of ERF genes in the TOBFAC database ends with NtERF240, the newly-uncovered gene is herein named NtERF241. The predicted coding sequence for NtERF241 was established by using the NtERF241 gene sequence and automated computational analysis programs available at the NCBI website.

### Example 2: NtERF241 Positively Regulates Nicotine Biosynthesis

This example demonstrates the use of NtERF241 or biologically active fragments thereof to positively regulate nicotine biosynthesis in plants and plant cell cultures.

### Methods

Plant and cell culture. Nicotiana tabacum cv. Burley 21 plants are grown as described in Reichers, D. E. and Timko, M. P., Plant Mol. Biol. 41:387-401 (1999). N. tabacum cv. Bright Yellow (BY-2) cell suspension cultures are grown in Murashige-Skoog (MS) medium containing 3% (w/v) sucrose and 0.2 mg/L 2.4-dichlorophenyoxyacetic acid (2.4-D), pH 5.8, and aliquots are transferred into fresh MS medium every 7 days to ensure that cells are maintained in the logarithmic growth phase. For MeJA treatments, cells are diluted into auxin-free media and grown at 28° C. for 1 day with shaking before treatment with 50 μM MeJA according to methods described by Xu and Timko, Plant Mol. Biol. 55:743-761 (2004). Three-week old plants are treated with 100 μM MeJA and collected 24 h after treatment.

Vector constructs. Expression vectors for overexpression analysis of NtERF241 alone or in combination with NtMYC1a, NtMYC1b, NtMYC2a, and/or NtMCY2b are prepared according to methods described in Sears et al., Plant Mol. Biol. 84:49-66 (2014). For RNAi knockdown studies, NtERF241-RNAi vectors are prepared according to the methods described in Sears et al. (2014).

Agrobacterium transformation of BY-2 cells. Transgene analysis (e.g., overexpression constructs, RNAi knockdown constructs) are performed in BY-2 cells transformed with Agrobacterium tumefaciens LBA4404 as described in Xu and Timko Plant Mol. Biol. 55:743-761 (2004) and Zhang et al. Mol. Plant 5:73-84 (2012). Transformed calli are selected on MS agar containing 50 mg/L kanamycin or 15 mg/L hygromycin (for RNAi vector) and 500 mg/L cefatoxime, and cell suspensions grown as described above.

Gene expression analysis. Total RNAs are isolated using Trizol reagent (Invitrogen) and reverse-transcribed with ThermoScript™ RT-PCR System (Invitrogen) according to the manufacturer's protocol. Semi-quantitative reverse transcription PCR (RT-PCR) assays are carried out using gene specific primer pairs in amplification run using the following conditions 96° C. for 1 min; 25 cycles of 94° C. for 30 s 58° C. for 30 s, 72° C. for 90 s; 72° C. for 10 min. The PCR products are separated on 2% agarose gel.

Quantitative RT-PCR (qRT-PCR) is performed as described by Zhang et al. (2012) using the iQ™ SYBR® Green Supermix (Bio-Rad).

Alkaloid analysis in BY-2 cells. Wild type or transgenic BY-2 cells are grown and subjected to MeJA treatment as described above. At 72 hours post-treatment, 0.5 g of cells are collected by vacuum-filtration, frozen in liquid nitrogen and lyophilized. Alkaloids are extracted from dried samples and measured by GCMS on a Shimadzu GCMS 2010 as described in Zhang et al. (2012).

### Results

It is predicted that plants and plant cell cultures genetically engineered to overexpress NtERF241 or biologically active fragments thereof will produce increased levels of nicotinic alkaloids as compared to non-transformed plants and plant cell cultures grown under similar conditions. It is further expected that the combined overexpression of NtERF241 and at least one additional transcription factor such as NtMYC1a, NtMYC1b, NtMYC2a, and NtMYC2b will have synergistic effects in this regard compared to that observed in plants or plant cell cultures overexpressing NtERF241 alone.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications, including GenBank Accession Numbers, referred to or cited herein are incorporated by reference in their entirety, including all figures and tables, to the extent they are not inconsistent with the explicit teachings of this specification.

Other embodiments are set forth within the following claims.

SEQUENCE LISTING

SEQ ID NO: 1 (1900 bp)
NtERF241 gene
GGCGCAAAGAGACTTTAATAAGAAATTACTTAAGTCAAGTTCTTGTGCTATATCTCGTTCGT
GATCCCGCCCTGGGAATCACATCACTGCCTTTTGACTGAAGTTCTTAGAAGAGGGAATGCAA
AAGAATTAGACTAGGGGCCTTATGCCAATCTTAATCCTTCCTTCACCCCGCCCTGGAATCAC
TGAACTCCCAAGACGTTATTTCTAATAGACCCTCAATCCGACTTATGTACTCCTTAAAAATT
AAGAAAAAGACAAAATTTTGGGGTAATAAATAATGGTAAATGGCTCCCGTCAATGAATGAAA
GTCGTTTTCTTATATTGGGCACGTGATAAAAGAACTTGGAAAAAAAGGCTTAAACAACTAGT
CCACTTGCCCAGCGAGAAACTTCAAAGAAGCGCAAATACTATTCCTTAATTAATTCAGACAT
TGAAAGTTTAATGAACTCCTTTTTTCAGGTGCATGTATCTTCATGCTCCACTGTTGTCTCTC
TTTTTCACTATATATACCCCAGTCCCTTCTTCTTTTTCAGAATTCTGACCCTTTTCCTCTTC
AATAACACAACACTCAAAAAATCCAATTATAACAAACAAAATGTATCAACCCATTTCTACAG
AATTCCCAGTATATCACCGGACTTCAAGTTTCAGTAGTCTCATGCCATGTTTGACGGATACT
TGGGGTGACTTGCCGTTAAAAGTTGATGATTCCGAAGATATGGTAATTTATGGGCTCTTAAG
TGACGCTTTAACTACCGGATGGACGCCGTTTAATTTAACGTCCACCGAAATAAAAGCCGAGC
CGAGGGAAGAGATTGAGCCAGCTACGAGTCCTGTTCCTTCAGTGGCTCCACCCGCGGAGACT
ACGACGGCTCAAGCCGTCGTGCCCAAGGGAAGGCATTATAGGGGCGTCAGGCAAAGGCCGTG
GGGGAAATTTGCGGCGGAAATAAGGGACCCAGCTAAAAATGGCGCACGGGTTTGGCTAGGGA
CTTATGAGACGGCTGAAGAAGCCGCGCTCGCTTATGATAAAGCAGCTTACAGGATGCGCGGC
TCCAAGGCTCTATTGAATTTTCCGCATAGGATCGGCTTAAATGAGCCTGAACCGGTTAGGCT
GACCGTTAAGAGACGATCACCTGAACCGGCCGTTAAGAGACGATCACCTGAACCGGCTAGCT
CGTCAATATCACCGGCTTCGGAAAATAGCTTGCCGAAGCGGAGGAGAAAAGCTGTAGCGGCT
AAGCAAGCTGAATTAGAAGTGCAGAGCCGATCAAATGTAATGCAAGTTGGGTGCCAAATGGA
ACAATTTCCAGTTGGCGAGCAGCTATTAGTTAGTTAAGATATGAGCTAAGAACTCAATTGTT
AAGTTTGGAGTGAATAGAAACAGCAAACTATTCCACTTTGCTTAGAGGTGGAGAGAGGCAGA
CCCAAGATTTGAGCACAACGGGGGCATCATTATTTTTAACATAAATATATAAGCTAGTAGCG
ATAAAATTTAGCGTGCTAACTCCTTCAAAATTTAATGATTATGAGTCAGTGATCAAAAATAT
CTTTTAAAATATCAAAACTTACTCAAATAAAATCAAGATTAAATATTCGTTAAGTAGTTCAA
GCAGAGTCTCAATCTCCATCGCTAAATCGACGGAGGTATGCTACTTTGCCGAAGTGATTTTT
GAAGGCACAAGCATTTTGGAGTTTTTTATCGCTCTTTTTAGGCGGAATTTTATTGAATTACT
TATTTTAATACAAGTCAAGAAAATGATATGCTTATAAACTTAGTTATCATGATAAACTTAGA
GAGAGACATATAAATTGGCTTCTTGCTAATGAAATATTTTATTCCTCTCTAATTTTCTTTAA
TCTTTTTATGTCTCTCTCTGTTACCTTTTTTAAATTCTAG
SEQ ID NO: 2 (741 bp)
NtERF241 ORF
ATGTATCAACCCATTTCTACAGAATTCCCAGTATATCACCGGACTTCAAGTTTCAGTAGTCT
CATGCCATGTTTGACGGATACTTGGGGTGACTTGCCGTTAAAAGTTGATGATTCCGAAGATA
TGGTAATTTATGGGCTCTTAAGTGACGCTTTAACTACCGGATGGACGCCGTTTAATTTAACG
TCCACCGAAATAAAAGCCGAGCCGAGGGAAGAGATTGAGCCAGCTACGAGTCCTGTTCCTTC
AGTGGCTCCACCCGCGGAGACTACGACGGCTCAAGCCGTCGTGCCCAAGGGAAGGCATTATA
GGGGCGTCAGGCAAAGGCCGTGGGGGAAATTTGCGGCGGAAATAAGGGACCCAGCTAAAAAT
GGCGCACGGGTTTGGCTAGGGACTTATGAGACGGCTGAAGAAGCCGCGCTCGCTTATGATAA
AGCAGCTTACAGGATGCGCGGCTCCAAGGCTCTATTGAATTTTCCGCATAGGATCGGCTTAA
ATGAGCCTGAACCGGTTAGGCTGACCGTTAAGAGACGATCACCTGAACCGGCCGTTAAGAGA
CGATCACCTGAACCGGCTAGCTCGTCAATATCACCGGCTTCGGAAAATAGCTTGCCGAAGCG
GAGGAGAAAAGCTGTAGCGGCTAAGCAAGCTGAATTAGAAGTGCAGAGCCGATCAAATGTAA
TGCAAGTTGGGTGCCAAATGGAACAATTTCCAGTTGGCGAGCAGCTATTAGTTAGTTAA
SEQ ID NO: 3 (246 AA)
NtERF241 polypeptide
MYQPISTEFPVYHRTSSFSSLMPCLTDTWGDLPLKVDDSEDMVIYGLLSDALTTGWTPFNLT
STEIKAEPREEIEPATSPVPSVAPPAETTTAQAVVPKGRHYRGVRQRPWGKFAAEIRDPAKN
GARVWLGTYETAEEAALAYDKAAYRMRGSKALLNFPHRIGLNEPEPVRLTVKRRSPEPAVKR
RSPEPASSSISPASENSLPKRRRKAVAAKQAELEVQSRSNVMQVGCQMEQFPVGEQLLVS
SEQ ID NO: 4 (2046 bp)
NtMYC1a ORF

| | |
|---|---|
| 1 | atgactgatt acagcttacc caccatgaat ttgtggaata ctagtggtac taccgatgac |
| 61 | aacgttacta tgatggaagc ttttatgtct tctgatctca cttcattttg ggctacttct |
| 121 | aattctactg ctgttgctgc tgttacctct aattctaatc atattccagt taatacccca |
| 181 | acggttcttc ttccgtcttc ttgtgcctct actgtcacag ctgtggctgt cgatgcttca |
| 241 | aaatccatgt cttttttcaa ccaagaaacc cttcaacagc gtcttcaaac gctcattgat |
| 301 | ggtgctcgtg agacgtggac ctatgccatc ttttggcagt catccgccgt tgatttaacg |
| 361 | agtccgtttg tgttgggctg gggagatggt tactacaaag gtgaagaaga taaagccaat |
| 421 | aggaaattag ctgtttcttc tcctgcttat atagctgagc aagaacaccg gaaaaaggtt |
| 481 | ctccgggagc tgaattcgtt gatttccggc acgcaaaccg gcactgatga tgccgtcgat |
| 541 | gaagaagtta ccgacactga atggttcttc cttatttcca tgacccagtc gtttgttaac |
| 601 | ggaagtgggc ttccgggtca ggccttatac aattccagcc ctatttgggt cgccggagca |
| 661 | gagaaattgg cagcttccca ctgcgaacgg gctcggcagg cccagggatt cgggcttcag |
| 721 | acgatggttt gtattccttc agcaaacggc gtggttgaat tgggctccac ggagttgatt |
| 781 | attcagagtt ctgatctcat gaacaaggtt agagtattgt ttaacttcaa taatgatttg |
| 841 | ggctctggtt cgtgggctgt gcaacccgag agcgatccgt ccgctctttg gctcactgat |
| 901 | ccatcgtctg cagctgtaca agtcaaagat ttaaatacag ttgaggcaaa ttcagttcca |
| 961 | tcaagtaata gtagtaagca agttgtattt gataatgaga ataatggtca cagttgtgat |
| 1021 | aatcagcaac agcaccattc tcggcaacaa acacaaggat tttttacaag ggagttgaac |
| 1081 | ttttcagaat tcgggtttga tggaagtagt aataatagga atgggaattc atcactttct |
| 1141 | tgcaagccag agtcggggga aatcttgaat tttggtgata gcactaagaa aagtgcaaat |
| 1201 | gggaacttat tttccggtca gtcccatttt ggtgcagggg aggagaataa gaagaagaaa |
| 1261 | aggtcacctg cttccagagg aagcaatgaa gaaggaatgc tttcatttgt ttcaggtaca |
| 1321 | atcttgcctg cagcttctgg tgcgatgaag tcaagtggat gtgtcggtga agactcctct |
| 1381 | gatcattcgg atcttgaggc ctcagtggtg aaagaagctg aaagtagtag agttgtagaa |
| 1441 | cccgaaaaga ggccaaagaa gcgaggaagg aagccagcaa atggacgtga ggaacctttg |
| 1501 | aatcacgtcg aagcagagag gcaaaggaga gagaaattaa accaaaggtt ctacgcttta |
| 1561 | agagctgttg ttccgaatgt gtccaagatg gacaaggcat cactgcttgg agatgcaatt |
| 1621 | tcatatatta atgagctgaa gttgaagctt caaactacag aaacagatag agaagacttg |
| 1681 | aagagccaaa tagaagattt gaagaaagaa ttagatagta aagactcaag gcgccctggt |
| 1741 | cctccaccac caaatcaaga tcacaagatg tctagccata ctggaagcaa gattgtagat |
| 1801 | gtggatatag atgttaagat aattggatgg gatgcgatga ttcgtataca atgtaataaa |
| 1861 | aagaaccatc cagctgcaag gttaatggta gccctcaagg agttagatct agatgtgcac |
| 1921 | catgccagtg tttcagtggt gaatgatttg atgatccaac aagccacagt gaaaatgggt |
| 1981 | agcagacttt acacggaaga gcaacttagg atagcattga catccagagt tgctgaaaca |
| 2041 | cgctaa |

SEQ ID NO: 5 (681 AA)
NtMYC1a polypeptide

| | |
|---|---|
| 1 | mtdyslptmn lwntsgttdd nvtmmeafms sdltsfwats nstavaavts nsnhipvntp |
| 61 | tvllpsscas tvtavavdas ksmsffnqet lqqrlqtlid garetwtyai fwqssavdlt |
| 121 | spfvlgwgdg yykgeedkan rklavsspay iaeqehrkkv lrelnslisg tqtgtddavd |
| 181 | eevtdtewff lismtqsfvn gsglpgqaly nsspiwvaga eklaashcer arqaqgfglq |
| 241 | tmvcipsang vvelgsteli iqssdlmnkv rvlfnfnndl gsgswavqpe sdpsalwltd |
| 301 | pssaavqvkd lntveansvp ssnsskqvvf dnennghscd nqqqhhsrqq tqgfftreln |
| 361 | fsefgfdgss nnrngnssls ckpesgeiln fgdstkksan gnlfsgqshf gageenkkkk |
| 421 | rspasrgsne egmlsfvsgt ilpaasgamk ssgcvgedss dhsdleasvv keaessrvve |
| 481 | pekrpkkrgr kpangreepl nhveaerqrr eklnqrfyal ravvpnvskm dkasllgdai |
| 541 | syinelklkl qttetdredl ksqiedlkke ldskdsrrpg ppppnqdhkm sshtgskivd |
| 601 | vdidvkiigw damiriqcnk knhpaarlmv alkeldldvh hasvsvvndl miqqatvkmg |
| 661 | srlyteeqlr ialtsrvaet r |

SEQ ID NO: 6 (2040 bp)
NtMYC1b ORF

| | |
|---|---|
| 1 | cgcagacccc tcttttcacc catttctctc tctctctctc tctctctctc tatatatata |
| 61 | tatatctttc acgccaccat atccaactgt ttgtgctggg tttatggaat gactgattac |
| 121 | agcttaccca ccatgaattt gtggaatact agtggtacta ccgatgacaa cgtttctatg |
| 181 | atggaatctt ttatgtcttc tgatctcact tcattttggg ctacttctaa ttctactact |
| 241 | gctgctgtta cctctaattc taatcttatt ccagttaata ccctaactgt tcttcttccg |
| 301 | tcttcttgtg cttctactgt cacagctgtg gctgtcgatg cttcaaaatc catgtctttt |
| 361 | ttcaaccaag aaactcttca gcagcgtctt caaaccctca ttgatggtgc tcgtgagacg |
| 421 | tggacctatg ccatcttttg gcagtcatcc gtcgttgatt tatcgagtcc gtttgtgttg |
| 481 | ggctggggag atggttacta caaaggtgaa gaagataaag ccaataggaa attagctgtt |
| 541 | tcttctcctg cttatattgc tgagcaagaa caccgaaaaa aggttctccg ggagctgaat |
| 601 | tcgttgatct ccggcacgca aaccggcact gatgatgccg tcgatgaaga agttaccgac |
| 661 | actgaatggt tcttccttat ttccatgacc caatcgtttg ttaacggaag tgggcttccg |
| 721 | ggtcaggcct tatacaattc cagccctatt tgggtcgccg gagcagagaa attggcagct |
| 781 | tcccactgcg aacgggctcg gcaggcccag ggattcgggc ttcagacgat ggtttgtatt |
| 841 | ccttcagcaa acggcgtggt tgaattgggc tccacggagt tgataatcca gagttgtgat |
| 901 | ctcatgaaca aggttagagt attgtttaac ttcaataatg atttgggctc tggttcgtgg |
| 961 | gctgtgcagc ccgagagcga tccgtccgct ctttggctca ctgatccatc gtctgcagct |
| 1021 | gtagaagtcc aagatttaaa tacagttaag gcaaattcag ttccatcaag taatagtagt |
| 1081 | aagcaagttg tgtttgataa tgagaataat ggtcacagtt ctgataatca gcaacagcag |
| 1141 | cattctaagc atgaaacaca aggatttttc acaagggagt tgaatttttc agaatttggg |
| 1201 | tttgatggaa gtagtaataa taggaatggg aattcatcac tttcttgcaa gccagagtcg |
| 1261 | ggggaaatct tgaattttgg tgatagtact aagaaaagtg caaatgggaa cttattttcg |
| 1321 | ggtcagtccc attttggggc aggggaggag aataagaaca agaaaaggtc acctgcttcc |
| 1381 | agaggaagca atgaagaagg aatgctttca tttgtttcgg gtacaatctt gcctgcagct |
| 1441 | tctggtgcga tgaagtcaag tggaggtgta ggtgaagact ctgatcattc ggatcttgag |
| 1501 | gcctcagtgg tgaaagaagc tgaaagtagt agagttgtag aacccgaaaa gaggccaaag |
| 1561 | aagcgaggaa ggaagccagc aaatggacgg gaggaacctt tgaatcacgt cgaagcagag |
| 1621 | aggcaaagga gagagaaatt aaaccaaagg ttctacgcat taagagctgt tgttccgaat |
| 1681 | gtgtccaaga tggacaaggc atcactgctt ggagatgcaa tttcatatat taatgagctg |
| 1741 | aagttgaagc ttcaaaatac agaaacagat agagaagaat tgaagagcca aatagaagat |
| 1801 | ttaaagaaag aattagttag taaagactca aggcgccctg gtcctccacc atcaaatcat |
| 1861 | gatcacaaga tgtctagcca tactggaagc aagattgtag acgtggatat agatgttaag |
| 1921 | ataattggat gggatgcgat gattcgtata caatgtaata aaaagaatca tccagctgca |
| 1981 | aggttaatgg tagccctcaa ggagttagat ctagatgtgc accatgccag tgtttcagtg |
| 2041 | gtgaacgatt tgatgatcca acaagccact gtgaaaatgg gtagcagact ttacacggaa |
| 2101 | gagcaactta ggatagcatt gacatccaga gttgctgaaa cacgctaa |

SEQ ID NO: 7 (679 AA)
NtMYC1b polypeptide

| | |
|---|---|
| 1 | mtdyslptmn lwntsgttdd nvsmmesfms sdltsfwats nsttaavtsn snlipvntlt |
| 61 | vllpsscast vtavavdask smsffnqett qqrlqtlidg aretwtyaif wqssvvdlss |
| 121 | pfvlgwgdgy ykgeedkanr klavsspayi aeqehrkkvl relnslisgt qtgtddavde |
| 181 | evtdtewffl ismtqsfvng sglpgqalyn sspiwvagae klaashcera rqaqgfglqt |
| 241 | mvcipsangv velgstelii qscdlmnkvr vlfnfnndlg sgswavqpes dpsalwltdp |
| 301 | ssaavevqdl ntvkansvps snsskqvvfd nennghssdn qqqqhskhet qgfftrelnf |
| 361 | sefgfdgssn nrngnsslsc kpesgeilnf gdstkksang nlfsgqshfg ageenknkkr |
| 421 | spasrgsnee gmlsfvsgti lpaasgamks sggvgedsdh sdleasvvke aessrvvepe |
| 481 | krpkkrgrkp angreeplnh veaerqrrek lnqrfyalra vvpnvskmdk asllgdaisy |
| 541 | inelklklqn tetdreelks qiedlkkelv skdsrrpgpp psnhdhkmss htgskivdvd |
| 601 | idvkiigwda miriqcnkkn hpaarlmval keldldvhha svsvvndlmi qqatvkmgsr |
| 661 | lyteeqlria ltsrvaetr |

SEQ ID NO: 8 (2214 bp)
NtMYC2a gene
CACACACTCTCTCCATTTTCACTCACTCCTTATCACCAAACAATTCTTGGGTGTTTGAATAT
ATACCCGAAATAATTTCCTCTCTGTATCAAGAATCAAACAGATCTGAATTGATTTGTCTGTT
TTTTTTTCTTGATTTTGTTATATGGAATGACGGATTATAGAATACCAACGATGACTAATATA
TGGAGCAATACTACATCCGATGATAATATGATGGAAGCTTTTTTATCTTCTGATCCGTCGTC
GTTTTGGCCCGGAACAACTACTACACCAACTCCCCGGAGTTCAGTTTCTCCAGCGCCGGCGC
CGGTGACGGGGATTGCCGGAGACCCATTAAAGTCTATGCCATATTTCAACCAAGAGTCACTG
CAACAGCGACTCCAGACTTTAATCGATGGGGCTCGCAAAGGGTGGACGTATGCCATATTTTG
GCAATCGTCTGTTGTGGATTTCGCGAGCCCCTCGGTTTTGGGGTGGGGAGATGGGTATTATA
AAGGTGAAGAAGATAAAAATAAGCGTAAAACGGCGTCGTTTTCGCCTGACTTTATCACGGAA
CAAGCACACCGGAAAAAGGTTCTCCGGGAGCTGAATTCTTTAATTTCCGGCACACAAACCGG
TGGTGAAAATGATGCTGTAGATGAAGAAGTAACTGATACTGAATGGTTTTTTCTGATTTCCA
TGACACAATCGTTTGTTAACGGAAGCGGGCTTCCGGGCCTGGCGATGTATAGTTCAAGCCCG
ATTTGGGTTACTGGAACAGAGAGATTAGCTGTTTCTCACTGTGAACGGGCCCGACAGGCCCA
AGGTTTCGGGCTTCAGACTATTGTTTGTATTCCTTCAGCTAATGGTGTTGTTGAGCTCGGGT
CAACTGAGTTGATATTCCAGACTGCTGATTTAATGAACAAGGTTAAAGTTTTGTTTAATTTT
AATATTGATATGGGTGCGACTACGGGCTCAGGATCGGGCTCATGTGCTATTCAGGCCGAGCC
CGATCCTTCAGCCCTTTGGCTGACTGATCCGGCTTCTTCAGTTGTGGAAGTCAAGGATTCGT
CGAATACAGTTCCTTCAAGGAATACCAGTAAGCAACTTGTGTTTGGAAATGAGAATTCTGAA
AATGGTAATCAAAATTCTCAGCAAACACAAGGATTTTTCACTAGGGAGTTGAATTTTTCCGA
ATATGGATTTGATGGAAGTAATACTCGGTATGGAAATGGGAATGCGAATTCTTCGCGTTCTT
GCAAGCCTGAGTCTGGTGAAATCTTGAATTTTGGTGATAGTACTAAGAGGAGTGCTTGCAGT
GCAAATGGGAGCTTGTTTTCGGGCCAATCACAGTTCGGGCCCGGGCCTGCGGAGGAGAACAA
GAACAAGAACAAGAAAAGGTCACCTGCATCAAGAGGAAGCAACGATGAAGGAATCCTTTCAT
TTGTTTCGGGTGTGATTTTGCCAAGTTCAAACACGGGGAAGTCCGGTGGAGGTGGCGATTCG
GATCAATCAGATCTCGAGGCTTCGGTGGTGAAGGAGGCGGATAGTAGTAGAGTTGTAGACCC
CGAGAAGAAGCCGAGGAAACGAGGGAGGAAACCGGCTAACGGGAGAGAGGAGCCATTGAATC
ATGTGGAGGCAGAGAGACAAAGGAGGGAGAAATTGAATCAAAGATTCTATGCACTTAGAGCT
GTTGTACCAAATGTGTCAAAAATGGATAAAGCATCACTTCTTGGTGATGCAATTGCATTTAT
CAATGAGTTGAAATCAAAGGTTCAGAATTCTGACTCAGATAAAGAGGACTTGAGGAACCAAA
TCGAATCTTTAAGGAATGAATTAGCCAACAAGGGATCAAACTATACCGGTCCTCCCCCGTCA
AATCAAGAACTCAAGATTGTAGATATGGACATCGACGTTAAGGTGATCGGATGGGATGCTAT
GATTCGTATACAATCTAATAAAAAGAACCATCCAGCCGCGAGGTTAATGACCGCTCTCATGG
AATTGGACTTAGATGTGCACCATGCTAGTGTTTCAGTTGTCAACGAGTTGATGATCCAACAA
GCGACTGTGAAAATGGGAAGCCGGCTTTACACGCAAGAACAACTTCGGATATCATTGACATC
CAGAATTGCTGAATCGCGATGAAGAGAAATACAGTAAATGGAAATTATCATAGTGAGCTCTG
AATAATGTTATCTTTCATTGAGCTATTTTAAGAGAATTTCTCCTAAAAAAAAAAAAAAAAAA
AAAAAAAAA
SEQ ID NO: 9 (659 AA)
NtMYC2a polypeptide

| | |
|---|---|
| 1 | mtdyriptmt niwsnttsdd nmmeaflssd pssfwpgttt tptprssvsp apapvtgiag |
| 61 | dplksmpyfn qeslqqrlqt lidgarkgwt yaifwqssvv dfaspsvlgw gdgyykgeed |
| 121 | knkrktasfs pdfiteqahr kkvlrelnsl isgtqtggen davdeevtdt ewfflismtq |
| 181 | sfvngsglpg lamyssspiw vtgterlays hcerargagg fglqtivcip sangvvelgs |
| 241 | telifqtadl mnkvkvlfnf nidmgattgs gsgscaiqae pdpsalwltd passvvevkd |
| 301 | ssntvpsrnt skqlvfgnen senvnqnsqg tqgfftreln fseygfdgsn trygngnans |
| 361 | srsckpesge ilnfgdstkr sacsangslf sgqsqfgpgp aeenknknkk rspasrgsnd |
| 421 | egilsfvsgv ilpssntgks ggggdsdqsd leasvvkead ssrvvdpekk prkrgrkpan |
| 481 | greeplnhve aerqrrekln qrfyalravv pnvskmdkas llgdaiafin elkskvqnsd |
| 541 | sdkedlrnqi eslrnelank gsnytgppps nqelkivdmd idvkvigwda miriqsnkkn |
| 601 | hpaarlmtal meldldvhha svsvvnelmi qqatvkmgsr lytqeqlris ltsriaesr |

SEQ ID NO: 10 (2391 bp)
NtMYC2b gene
GTAACAAACCCTCTCCATTTTCACTCACTCCAAAAAACTTTCCTCTCTATTTTTTCTCTCTG
TATCAAGAATCAAACAGATCTGAATTGATTTGGGAGTTTTTTTTCTTCTTGTTTTTGTTATA
TGGAATGACGGACTATAGAATACCAACGATGACTAATATATGGAGCAATACAACATCCGACG
ATAACATGATGGAAGCTTTTTTATCTTCTGATCCGTCGTCGTTTTGGGCCGGAACAAATACA
CCAACTCCACGGAGTTCAGTTTCTCCGGCGCCGGCGCCGGTGACGGGGATTGCCGGAGACCC
ATTAAAGTCGATGCCGTATTTCAACCAAGAGTCGCTGCAACAGCGACTCCAGACGTTAATCG
ACGGGGCTCGCGAAGCGTGGACTTACGCCATATTCTGGCAATCGTCTGTTGTGGATTTCGTG
AGCCCCTCGGTGTTGGGGTGGGGAGATGGATATTATAAAGGAGAAGAAGACAAGAATAAGCG
TAAAACGGCGGCGTTTTCGCCTGATTTTATTACGGAGCAAGAACACCGGAAAAAAGTTCTCC
GGGAGCTGAATTCTTTAATTTCCGGCACACAAACTGGTGGTGAAAATGATGCTGTAGATGAA
GAAGTAACGGATACTGAATGGTTTTTTCTGATTTCAATGACTCAATCGTTTGTTAACGGAAG
CGGGCTTCCGGGCCTGGCTATGTACAGCTCAAGCCCGATTTGGGTTACTGGAAGAGAAAGAT
TAGCTGCTTCTCACTGTGAACGGGCCCGACAGGCCCAAGGTTTCGGGCTTCAGACTATGGTT
TGTATTCCTTCAGCTAATGGTGTTGTTGAGCTCGGGTCAACTGAGTTGATATTCCAGAGCGC
TGATTTAATGAACAAGGTTAAAATCTTGTTTGATTTTAATATTGATATGGGCGCGACTACGG
GCTCAGGTTCGGGCTCATGTGCTATTCAGGCTGAGCCCGATCCTTCAACCCTTTGGCTTACG
GATCCACCTTCCTCAGTTGTGGAAGTCAAGGATTCGTCGAATACAGTTCCTTCAAGTAATAG
TAGTAAGCAACTTGTGTTTGGAAATGAGAATTCTGAAAATGTTAATCAAAATTCTCAGCAAA
CACAAGGATTTTTCACTAGGGAGTTGAATTTTTCCGAATATGGATTTGATGGAAGTAATACT
AGGAGTGGAAATGGGAATGTGAATTCTTCGCGTTCTTGCAAGCCTAGAAATGCTTCAAGTGC
AAATGGGAGCTTGTTTTCGGGCCAATCGCAGTTCGGTCCCGGGCCTGCGGAGGAGAACAAGA
ACAAGAACAAGAAAAGGTCACCTGCATCAAGAGGAAGCAATGAAGAAGGAATGCTTTCATTT
GTTTCGGGTGTGATCTTGCCAAGTTCAAACACGGGGAAGTCCGGTGGAGGTGGCGATTCGGA
TCATTCAGATCTCGAGGCTTCGGTGGTGAAGGAGGCGGATAGTAGTAGAGTTGTAGACCCCG
AGAAGAGGCCGAGGAAACGAGGAAGGAAACCGGCTAACGGGAGAGAGGAGCCATTGAATCAT
GTGGAGGCAGAGAGGCAAAGGAGGGAGAAATTGAATCAAAGATTCTATGCACTTAGAGCTGT
TGTACCAAATGTGTCAAAAATGGATAAAGCATCACTTCTTGGTGATGCAATTGCATTTATCA
ATGAGTTGAAATCAAAGGTTGAGAATTCTGAGTCAGATAAAGATGAGTTGAGGAACCAAATT
GAATCTTTAAGGAATGAATTAGCCAACAAGGGATCAAACTATACCGGTCCTCCACCGCCAAA
TCAAGATCTCAAGATTGTAGATATGGATATCGACGTTAAAGTCATCGGATGGGATGCTATGA
TTCGTATACAATCTAATAAAAAGAACCATCCAGCCGCGAGGTTAATGGCCGCTCTCATGGAA
TTGGACTTAGATGTGGACCATGCTAGTGTTTGAGTTGTCAACGAGTTGATGATCCAACAAGC
GACAGTGAAAATGGGGAGCCGGCTTTACACGGAAGAGCAGCTTGGGATATCATTGACATCCA
GAATTGCTGAATCGCGATGAAGAGAAATACAGTAAATGGAAATTATTAGTGAGCTCTGAATA
ATGTTATCTTTCATTGAGCTATTTTAAGAGAATTTCTCCTATAGTTAGATCTTGAGATTAAG
GCTACTTAAAAGTGGAAAGTTGATTGAGCTTTCCTCTTAGTTTTTTGGGTATTTTTCAACTT
TTATATCTAGTTTGTTTTCCACATTTTCTGTACATATAATGTGAAACCAATACTAGATCTCA
AGATCTGGTTTTTAGTTCTGTAATTAGAAATAAATATGCAGCTTCATCTTTTTCTGTTAAAA
AAAAAAAAAAAAAAAAAAAAA
SEQ ID NO: 11 (658 AA)
NtMYC2b polypeptide

| | |
|---|---|
| 1 | mtdyriptmt niwsnttsdd nmmeaflssd pssfwagtnt ptprssvspa papvtgiagd |
| 61 | plksmpyfnq eslqqrlqtl idgareawty aifwqssvvd fvspsvlgwg dgyykgeedk |
| 121 | nkrktaafsp dfiteqehrk kvlrelnsli sgtqtggend avdeevtdte wfflismtqs |
| 181 | fvngsglpgl amyssspiwv tgrerlaash cerargaggf glqtmvcips angvvelgst |
| 241 | elifqsadlm nkvkilfdfn idmgattgsg sgscaiqaep dpstlwltdp pssvvevkds |
| 301 | sntvpssnss kqlvfgnens envnqnsqqt qgfftrelnf seygfdgsnt rsgngnvnss |
| 361 | rsckpesgei lnfgdstkrn assangslfs gqsqfgpgpa eenknknkkr spasrgsnee |
| 421 | gmlsfvsgvi lpssntgksg gggdsdhsdl easvvkeads srvvdpekrp rkrgrkpang |
| 481 | reeplnhvea erqrreklnq rfyalravvp nvskmdkasl lgdaiafine lkskvqnsds |
| 541 | dkdelrnqie slrnelankg snytgppppn qdlkivdmdi dvkvigwdam iriqsnkknh |
| 601 | paarlmaalm eldldvhhas vsvvnelmiq qatvkmgsrl ytqeqlrisl tsriaesr |

1. An isolated cDNA molecule comprising a nucleotide sequence selected from the group consisting of:
(a) a nucleotide sequence set forth in SEQ ID NO: 2;
(b) a nucleotide sequence that encodes a polypeptide having the amino acid sequence set forth in SEQ ID NO: 3; and
(c) a nucleotide sequence that is at least about 90% identical to the nucleotide sequences of (a) or (b), and which encodes a transcription factor that positively regulates nicotinic alkaloid biosynthesis,
wherein the nucleotide sequence is operably linked to a heterologous nucleic acid.

2. An expression vector comprising the cDNA molecule of claim 1, operably linked to one or more control sequences suitable for directing expression in a Nicotiana host cell.

3. A genetically engineered nicotinic alkaloid-producing Nicotiana plant comprising a cell comprising a chimeric nucleic acid construct comprising the isolated cDNA molecule of claim 1.

4. The engineered Nicotiana plant of claim 3, wherein the plant is a Nicotiana tabacum plant.

5. Seeds from the engineered Nicotiana plant of claim 3, wherein the seeds comprise the chimeric nucleic acid construct.

6. A tobacco product comprising the engineered Nicotiana plant of claim 3.

7.-10. (canceled)

11. A method for increasing a nicotinic alkaloid in a Nicotiana plant, comprising:
(a) introducing into a Nicotiana plant an expression vector comprising the nucleotide sequence of claim 1;
and
(b) growing the plant under conditions which allow for the expression of a transcription factor that positively regulates nicotinic alkaloid biosynthesis from the nucleotide sequence;
wherein expression of the transcription factor results in the plant having an increased nicotinic alkaloid content as compared to a control plant grown under similar conditions.

12.-20. (canceled)

21. A method for reducing a nicotinic alkaloid in a Nicotiana plant, comprising down-regulating a transcription factor that positively regulates alkaloid biosynthesis, wherein the transcription factor is down-regulated by:
(a) introducing into a Nicotiana plant cell a nucleic acid comprising at least about 15 consecutive nucleotides of the cDNA molecule comprising the nucleotide sequence of claim 1;
wherein the consecutive nucleotides are in sense orientation, antisense orientation, or both;
(b) producing a plant comprising the plant cell; and
(c) growing the plant under conditions whereby the nucleotide sequence decreases levels of the transcription factor in the plant as compared to a control plant grown under similar conditions.

22. (canceled)

23. A method for reducing a nicotinic alkaloid in a Nicotiana plant, comprising down-regulating a transcription factor that positively regulates alkaloid biosynthesis, wherein the transcription factor is down-regulated by:
(a) introducing into a population of plant cells a reagent for site-directed mutagenesis of a target comprising at least about 15 consecutive nucleotides of a cDNA molecule comprising a nucleotide sequence selected from the group consisting of:
(i) a nucleotide sequence set forth in SEQ ID NO: 2;
(ii) a nucleotide sequence that encodes a polypeptide having the amino acid sequence set forth in SEQ ID NO: 3; and
(iii) a nucleotide sequence that is at least about 90% identical to the nucleotide sequences of (i) or (ii), and which encodes a transcription factor that positively regulates alkaloid biosynthesis; and
(b) detecting and selecting a target mutated plant cell or a plant derived from such a cell, wherein the target mutated plant cell or plant has a mutation in a gene encoding transcription factor positively regulating alkaloid biosynthesis and reduced alkaloid content as compared to a control plant.

24. The method of claim 23, wherein the reagent is a recombinagenic oligonucleobase or a targeted nuclease.

25. (canceled)

26. A mutated plant produced by the method of claim 23, wherein the plant has reduced expression of a transcription factor that positively regulates nicotinic alkaloid biosynthesis and reduced alkaloid content as compared to a control plant.

27. A product comprising the mutated plant of claim 26 or portions thereof, wherein the product has a reduced level of a nicotinic alkaloid as compared to a product produced from a control plant.

28. Seeds from the mutated plant of claim 26.

29. A method for reducing nicotinic alkaloid levels in a population of Nicotiana plants, comprising:
(a) providing a population of mutated Nicotiana plants;
(b) detecting and selecting a target mutated plant within the population, wherein (i) the target mutated plant has decreased expression of a transcription factor that positively regulates alkaloid biosynthesis as compared to a control plant, (ii) the detection comprises using a cDNA molecule as a primer or a probe, and (iii) the cDNA molecule comprises a nucleotide sequence selected from the group consisting of:
(1) a nucleotide sequence set forth in SEQ ID NO: 2;
(2) a nucleotide sequence that encodes a polypeptide having the amino acid sequence set forth in SEQ ID NO: 3; and
(3) a nucleotide sequence that is at least about 90% identical to the nucleotide sequences of (1) or (2), and which encodes a transcription factor that positively regulates alkaloid biosynthesis; and
(c) selectively breeding the target mutated plant to produce a population of plants having decreased expression of a transcription factor that positively regulates alkaloid biosynthesis as compared to a population of control plants.

30. A mutated alkaloid-producing Nicotiana plant produced by the method of claim 29, wherein the plant has reduced expression of a transcription factor that positively regulates alkaloid biosynthesis and reduced alkaloid content, as compared to a control plant.

31. (canceled)

32. A tobacco product comprising the mutated plant of claim 30 or portions thereof, wherein the product has a reduced level of a nicotinic alkaloid as compared to a product produced from a control plant.

33. (canceled)

34. A genetically engineered tobacco plant overexpressing a gene product encoded by SEQ ID NO: 2, wherein the genetically engineered plant exhibits increased expression of the gene product as compared to a control and the genetically engineered plant comprises cells comprising a nucleic acid construct comprising in the 5′ to 3′ direction:
(a) a promoter operable in the plant cell, and
(b) a heterologous nucleotide sequence operably associated with the promoter, wherein the heterologous nucleotide sequence comprises the nucleotide sequence set forth in SEQ ID NO: 2.

35. Progeny of the genetically engineered plant according to claim 34, wherein the progeny have overexpression of a gene product encoded by SEQ ID NO: 2.

36. A method of making a genetically engineered increased-nicotine tobacco cell having overexpression of a gene product encoded by SEQ ID NO: 2, the method comprising introducing the cDNA molecule of claim 1 into the cell to genetically engineer overexpression of a gene product encoded by SEQ ID NO 2.

37. The method of claim 36, further comprising genetically engineering overexpression within the tobacco cell of at least one additional transcription factor that positively regulates nicotinic alkaloid biosynthesis, wherein the additional transcription factor that positively regulates nicotinic alkaloid biosynthesis is at least one of NtMYC1a, NtMYC1b, NtMYC2a, or NtMYC2b.

38. (canceled)

39. The method of claim 36, further comprising genetically engineering overexpression within the tobacco cell of one or more nicotinic alkaloid biosynthesis enzymes selected from the group consisting of NBB1, A622, quinolate phosphoribosyltransferase (QPT), putrescine-N-methyltransferase (PMT), or N-methylputrescine oxidase (MPO).

40. A tobacco plant cell produced by the method of claim 36.


SEQUENCE LISTING (11 sequences)

SEQ ID NO: 1 - DNA, 1900 bp, Nicotiana tabacum
ggcgcaaagagactttaataagaaattacttaagtcaagttcttgtgctatatctcgttcgtgatcccgccctgggaatcacatcactgccttttgactgaagttcttagaagagggaatgcaaaagaattagactaggggccttatgccaatcttaatccttccttcaccccgccctggaatcactgaactcccaagacgttatttctaatagaccctcaatccgacttatgtactccttaaaaattaagaaaaagacaaaattttggggtaataaataatggtaaatggctcccgtcaatgaatgaaagtcgttttcttatattgggcacgtgataaaagaacttggaaaaaaaggcttaaacaactagtccacttgcccagcgagaaacttcaaagaagcgcaaatactattccttaattaattcagacattgaaagtttaatgaactccttttttcaggtgcatgtatcttcatgctccactgttgtctctctttttcactatatataccccagtcccttcttctttttcagaattctgacccttttcctcttcaataacacaacactcaaaaaatccaattataacaaacaaaatgtatcaacccatttctacagaattcccagtatatcaccggacttcaagtttcagtagtctcatgccatgtttgacggatacttggggtgacttgccgttaaaagttgatgattccgaagatatggtaatttatgggctcttaagtgacgctttaactaccggatggacgccgtttaatttaacgtccaccgaaataaaagccgagccgagggaagagattgagccagctacgagtcctgttccttcagtggctccacccgcggagactacgacggctcaagccgtcgtgcccaagggaaggcattataggggcgtcaggcaaaggccgtgggggaaatttgcggcggaaataagggacccagctaaaaatggcgcacgggtttggctagggacttatgagacggctgaagaagccgcgctcgcttatgataaagcagcttacaggatgcgcggctccaaggctctattgaattttccgcataggatcggcttaaatgagcctgaaccggttaggctgaccgttaagagacgatcacctgaaccggccgttaagagacgatcacctgaaccggctagctcgtcaatatcaccggcttcggaaaatagcttgccgaagcggaggagaaaagctgtagcggctaagcaagctgaattagaagtgcagagccgatcaaatgtaatgcaagttgggtgccaaatggaacaatttccagttggcgagcagctattagttagttaagatatgagctaagaactcaattgttaagtttggagtgaatagaaacagcaaactattccactttgcttagaggtggagagaggcagacccaagatttgagcacaacgggggcatcattatttttaacataaatatataagctagtagcgataaaatttagcgtgctaactccttcaaaatttaatgattatgagtcagtgatcaaaaatatcttttaaaatatcaaaacttactcaaataaaatcaagattaaatattcgttaagtagttcaagcagagtctcaatctccatcgctaaatcgacggaggtatgctactttgccgaagtgatttttgaaggcacaagcattttggagttttttatcgctctttttaggcggaattttattgaattacttattttaatacaagtcaagaaaatgatatgcttataaacttagttatcatgataaacttagagagagacatataaattggcttcttgctaatgaaatattttattcctctctaattttctttaatctttttatgtctctctctgttaccttttttaaattctag

SEQ ID NO: 2 - DNA, 741 bp, Nicotiana tabacum
atgtatcaacccatttctacagaattcccagtatatcaccggacttcaagtttcagtagtctcatgccatgtttgacggatacttggggtgacttgccgttaaaagttgatgattccgaagatatggtaatttatgggctcttaagtgacgctttaactaccggatggacgccgtttaatttaacgtccaccgaaataaaagccgagccgagggaagagattgagccagctacgagtcctgttccttcagtggctccacccgcggagactacgacggctcaagccgtcgtgcccaagggaaggcattataggggcgtcaggcaaaggccgtgggggaaatttgcggcggaaataagggacccagctaaaaatggcgcacgggtttggctagggacttatgagacggctgaagaagccgcgctcgcttatgataaagcagcttacaggatgcgcggctccaaggctctattgaattttccgcataggatcggcttaaatgagcctgaaccggttaggctgaccgttaagagacgatcacctgaaccggccgttaagagacgatcacctgaaccggctagctcgtcaatatcaccggcttcggaaaatagcttgccgaagcggaggagaaaagctgtagcggctaagcaagctgaattagaagtgcagagccgatcaaatgtaatgcaagttgggtgccaaatggaacaatttccagttggcgagcagctattagttagttaa

SEQ ID NO: 3 - protein, 246 aa, Nicotiana tabacum
MYQPISTEFPVYHRTSSFSSLMPCLTDTWGDLPLKVDDSEDMVIYGLLSDALTTGWTPFNLTSTEIKAEPREEIEPATSPVPSVAPPAETTTAQAVVPKGRHYRGVRQRPWGKFAAEIRDPAKNGARVWLGTYETAEEAALAYDKAAYRMRGSKALLNFPHRIGLNEPEPVRLTVKRRSPEPAVKRRSPEPASSSISPASENSLPKRRRKAVAAKQAELEVQSRSNVMQVGCQMEQFPVGEQLLVS

SEQ ID NO: 4 - DNA, 2046 bp, Nicotiana tabacum
atgactgattacagcttacccaccatgaatttgtggaatactagtggtactaccgatgacaacgttactatgatggaagcttttatgtcttctgatctcacttcattttgggctacttctaattctactgctgttgctgctgttacctctaattctaatcatattccagttaataccccaacggttcttcttccgtcttcttgtgcctctactgtcacagctgtggctgtcgatgcttcaaaatccatgtcttttttcaaccaagaaacccttcaacagcgtcttcaaacgctcattgatggtgctcgtgagacgtggacctatgccatcttttggcagtcatccgccgttgatttaacgagtccgtttgtgttgggctggggagatggttactacaaaggtgaagaagataaagccaataggaaattagctgtttcttctcctgcttatatagctgagcaagaacaccggaaaaaggttctccgggagctgaattcgttgatttccggcacgcaaaccggcactgatgatgccgtcgatgaagaagttaccgacactgaatggttcttccttatttccatgacccagtcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgattattcagagttctgatctcatgaacaaggttagagtattgtttaacttcaataatgatttgggctctggttcgtgggctgtgcaacccgagagcgatccgtccgctctttggctcactgatccatcgtctgcagctgtacaagtcaaagatttaaatacagttgaggcaaattcagttccatcaagtaatagtagtaagcaagttgtatttgataatgagaataatggtcacagttgtgataatcagcaacagcaccattctcggcaacaaacacaaggattttttacaagggagttgaacttttcagaattcgggtttgatggaagtagtaataataggaatgggaattcatcactttcttgcaagccagagtcgggggaaatcttgaattttggtgatagcactaagaaaagtgcaaatgggaacttattttccggtcagtcccattttggtgcaggggaggagaataagaagaagaaaaggtcacctgcttccagaggaagcaatgaagaaggaatgctttcatttgtttcaggtacaatcttgcctgcagcttctggtgcgatgaagtcaagtggatgtgtcggtgaagactcctctgatcattcggatcttgaggcctcagtggtgaaagaagctgaaagtagtagagttgtagaacccgaaaagaggccaaagaagcgaggaaggaagccagcaaatggacgtgaggaacctttgaatcacgtcgaagcagagaggcaaaggagagagaaattaaaccaaaggttctacgctttaagagctgttgttccgaatgtgtccaagatggacaaggcatcactgcttggagatgcaatttcatatattaatgagctgaagttgaagcttcaaactacagaaacagatagagaagacttgaagagccaaatagaagatttgaagaaagaattagatagtaaagactcaaggcgccctggtcctccaccaccaaatcaagatcacaagatgtctagccatactggaagcaagattgtagatgtggatatagatgttaagataattggatgggatgcgatgattcgtatacaatgtaataaaaagaaccatccagctgcaaggttaatggtagccctcaaggagttagatctagatgtgcaccatgccagtgtttcagtggtgaatgatttgatgatccaacaagccacagtgaaaatgggtagcagactttacacggaagagcaacttaggatagcattgacatccagagttgctgaaacacgctaa

SEQ ID NO: 5 - protein, 681 aa, Nicotiana tabacum
MTDYSLPTMNLWNTSGTTDDNVTMMEAFMSSDLTSFWATSNSTAVAAVTSNSNHIPVNTPTVLLPSSCASTVTAVAVDASKSMSFFNQETLQQRLQTLIDGARETWTYAIFWQSSAVDLTSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSSDLMNKVRVLFNFNNDLGSGSWAVQPESDPSALWLTDPSSAAVQVKDLNTVEANSVPSSNSSKQVVFDNENNGHSCDNQQQHHSRQQTQGFFTRELNFSEFGFDGSSNNRNGNSSLSCKPESGEILNFGDSTKKSANGNLFSGQSHFGAGEENKKKKRSPASRGSNEEGMLSFVSGTILPAASGAMKSSGCVGEDSSDHSDLEASVVKEAESSRVVEPEKRPKKRGRKPANGREEPLNHVEAERQRREKLNQRFYALRAVVPNVSKMDKASLLGDAISYINELKLKLQTTETDREDLKSQIEDLKKELDSKDSRRPGPPPPNQDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 6 - DNA, 2148 bp, Nicotiana tabacum
cgcagacccctcttttcacccatttctctctctctctctctctctctctctatatatatatatatctttcacgccaccatatccaactgtttgtgctgggtttatggaatgactgattacagcttacccaccatgaatttgtggaatactagtggtactaccgatgacaacgtttctatgatggaatcttttatgtcttctgatctcacttcattttgggctacttctaattctactactgctgctgttacctctaattctaatcttattccagttaataccctaactgttcttcttccgtcttcttgtgcttctactgtcacagctgtggctgtcgatgcttcaaaatccatgtcttttttcaaccaagaaactcttcagcagcgtcttcaaaccctcattgatggtgctcgtgagacgtggacctatgccatcttttggcagtcatccgtcgttgatttatcgagtccgtttgtgttgggctggggagatggttactacaaaggtgaagaagataaagccaataggaaattagctgtttcttctcctgcttatattgctgagcaagaacaccgaaaaaaggttctccgggagctgaattcgttgatctccggcacgcaaaccggcactgatgatgccgtcgatgaagaagttaccgacactgaatggttcttccttatttccatgacccaatcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgataatccagagttgtgatctcatgaacaaggttagagtattgtttaacttcaataatgatttgggctctggttcgtgggctgtgcagcccgagagcgatccgtccgctctttggctcactgatccatcgtctgcagctgtagaagtccaagatttaaatacagttaaggcaaattcagttccatcaagtaatagtagtaagcaagttgtgtttgataatgagaataatggtcacagttctgataatcagcaacagcagcattctaagcatgaaacacaaggatttttcacaagggagttgaatttttcagaatttgggtttgatggaagtagtaataataggaatgggaattcatcactttcttgcaagccagagtcgggggaaatcttgaattttggtgatagtactaagaaaagtgcaaatgggaacttattttcgggtcagtcccattttggggcaggggaggagaataagaacaagaaaaggtcacctgcttccagaggaagcaatgaagaaggaatgctttcatttgtttcgggtacaatcttgcctgcagcttctggtgcgatgaagtcaagtggaggtgtaggtgaagactctgatcattcggatcttgaggcctcagtggtgaaagaagctgaaagtagtagagttgtagaacccgaaaagaggccaaagaagcgaggaaggaagccagcaaatggacgggaggaacctttgaatcacgtcgaagcagagaggcaaaggagagagaaattaaaccaaaggttctacgcattaagagctgttgttccgaatgtgtccaagatggacaaggcatcactgcttggagatgcaatttcatatattaatgagctgaagttgaagcttcaaaatacagaaacagatagagaagaattgaagagccaaatagaagatttaaagaaagaattagttagtaaagactcaaggcgccctggtcctccaccatcaaatcatgatcacaagatgtctagccatactggaagcaagattgtagacgtggatatagatgttaagataattggatgggatgcgatgattcgtatacaatgtaataaaaagaatcatccagctgcaaggttaatggtagccctcaaggagttagatctagatgtgcaccatgccagtgtttcagtggtgaacgatttgatgatccaacaagccactgtgaaaatgggtagcagactttacacggaagagcaacttaggatagcattgacatccagagttgctgaaacacgctaa

SEQ ID NO: 7 - protein, 679 aa, Nicotiana tabacum
MTDYSLPTMNLWNTSGTTDDNVSMMESFMSSDLTSFWATSNSTTAAVTSNSNLIPVNTLTVLLPSSCASTVTAVAVDASKSMSFFNQETLQQRLQTLIDGARETWTYAIFWQSSVVDLSSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSCDLMNKVRVLFNFNNDLGSGSWAVQPESDPSALWLTDPSSAAVEVQDLNTVKANSVPSSNSSKQVVFDNENNGHSSDNQQQQHSKHETQGFFTRELNFSEFGFDGSSNNRNGNSSLSCKPESGEILNFGDSTKKSANGNLFSGQSHFGAGEENKNKKRSPASRGSNEEGMLSFVSGTILPAASGAMKSSGGVGEDSDHSDLEASVVKEAESSRVVEPEKRPKKRGRKPANGREEPLNHVEAERQRREKLNQRFYALRAVVPNVSKMDKASLLGDAISYINELKLKLQNTETDREELKSQIEDLKKELVSKDSRRPGPPPSNHDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 8 - DNA, 2241 bp, Nicotiana tabacum
cacacactctctccattttcactcactccttatcaccaaacaattcttgggtgtttgaatatatacccgaaataatttcctctctgtatcaagaatcaaacagatctgaattgatttgtctgtttttttttcttgattttgttatatggaatgacggattatagaataccaacgatgactaatatatggagcaatactacatccgatgataatatgatggaagcttttttatcttctgatccgtcgtcgttttggcccggaacaactactacaccaactccccggagttcagtttctccagcgccggcgccggtgacggggattgccggagacccattaaagtctatgccatatttcaaccaagagtcactgcaacagcgactccagactttaatcgatggggctcgcaaagggtggacgtatgccatattttggcaatcgtctgttgtggatttcgcgagcccctcggttttggggtggggagatgggtattataaaggtgaagaagataaaaataagcgtaaaacggcgtcgttttcgcctgactttatcacggaacaagcacaccggaaaaaggttctccgggagctgaattctttaatttccggcacacaaaccggtggtgaaaatgatgctgtagatgaagaagtaactgatactgaatggttttttctgatttccatgacacaatcgtttgttaacggaagcgggcttccgggcctggcgatgtatagttcaagcccgatttgggttactggaacagagagattagctgtttctcactgtgaacgggcccgacaggcccaaggtttcgggcttcagactattgtttgtattccttcagctaatggtgttgttgagctcgggtcaactgagttgatattccagactgctgatttaatgaacaaggttaaagttttgtttaattttaatattgatatgggtgcgactacgggctcaggatcgggctcatgtgctattcaggccgagcccgatccttcagccctttggctgactgatccggcttcttcagttgtggaagtcaaggattcgtcgaatacagttccttcaaggaataccagtaagcaacttgtgtttggaaatgagaattctgaaaatggtaatcaaaattctcagcaaacacaaggatttttcactagggagttgaatttttccgaatatggatttgatggaagtaatactcggtatggaaatgggaatgcgaattcttcgcgttcttgcaagcctgagtctggtgaaatcttgaattttggtgatagtactaagaggagtgcttgcagtgcaaatgggagcttgttttcgggccaatcacagttcgggcccgggcctgcggaggagaacaagaacaagaacaagaaaaggtcacctgcatcaagaggaagcaacgatgaaggaatcctttcatttgtttcgggtgtgattttgccaagttcaaacacggggaagtccggtggaggtggcgattcggatcaatcagatctcgaggcttcggtggtgaaggaggcggatagtagtagagttgtagaccccgagaagaagccgaggaaacgagggaggaaaccggctaacgggagagaggagccattgaatcatgtggaggcagagagacaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagaggacttgaggaaccaaatcgaatctttaaggaatgaattagccaacaagggatcaaactataccggtcctcccccgtcaaatcaagaactcaagattgtagatatggacatcgacgttaaggtgatcggatgggatgctatgattcgtatacaatctaataaaaagaaccatccagccgcgaggttaatgaccgctctcatggaattggacttagatgtgcaccatgctagtgtttcagttgtcaacgagttgatgatccaacaagcgactgtgaaaatgggaagccggctttacacgcaagaacaacttcggatatcattgacatccagaattgctgaatcgcgatgaagagaaatacagtaaatggaaattatcatagtgagctctgaataatgttatctttcattgagctattttaagagaatttctcctaaaaaaaaaaaaaaaaaaaaaaaaaaa

SEQ ID NO: 9 - protein, 659 aa, Nicotiana tabacum
MTDYRIPTMTNIWSNTTSDDNMMEAFLSSDPSSFWPGTTTTPTPRSSVSPAPAPVTGIAGDPLKSMPYFNQESLQQRLQTLIDGARKGWTYAIFWQSSVVDFASPSVLGWGDGYYKGEEDKNKRKTASFSPDFITEQAHRKKVLRELNSLISGTQTGGENDAVDEEVTDTEWFFLISMTQSFVNGSGLPGLAMYSSSPIWVTGTERLAVSHCERARQAQGFGLQTIVCIPSANGVVELGSTELIFQTADLMNKVKVLFNFNIDMGATTGSGSGSCAIQAEPDPSALWLTDPASSVVEVKDSSNTVPSRNTSKQLVFGNENSENVNQNSQQTQGFFTRELNFSEYGFDGSNTRYGNGNANSSRSCKPESGEILNFGDSTKRSACSANGSLFSGQSQFGPGPAEENKNKNKKRSPASRGSNDEGILSFVSGVILPSSNTGKSGGGGDSDQSDLEASVVKEADSSRVVDPEKKPRKRGRKPANGREEPLNHVEAERQRREKLNQRFYALRAVVPNVSKMDKASLLGDAIAFINELKSKVQNSDSDKEDLRNQIESLRNELANKGSNYTGPPPSNQELKIVDMDIDVKVIGWDAMIRIQSNKKNHPAARLMTALMELDLDVHHASVSVVNELMIQQATVKMGSRLYTQEQLRISLTSRIAESR

SEQ ID NO: 10 - DNA, 2377 bp, Nicotiana tabacum
gtaacaaaccctctccattttcactcactccaaaaaactttcctctctattttttctctctgtatcaagaatcaaacagatctgaattgatttgggagttttttttcttcttgtttttgttatatggaatgacggactatagaataccaacgatgactaatatatggagcaatacaacatccgacgataacatgatggaagcttttttatcttctgatccgtcgtcgttttgggccggaacaaatacaccaactccacggagttcagtttctccggcgccggcgccggtgacggggattgccggagacccattaaagtcgatgccgtatttcaaccaagagtcgctgcaacagcgactccagacgttaatcgacggggctcgcgaagcgtggacttacgccatattctggcaatcgtctgttgtggatttcgtgagcccctcggtgttggggtggggagatggatattataaaggagaagaagacaagaataagcgtaaaacggcggcgttttcgcctgattttattacggagcaagaacaccggaaaaaagttctccgggagctgaattctttaatttccggcacacaaactggtggtgaaaatgatgctgtagatgaagaagtaacggatactgaatggttttttctgatttcaatgactcaatcgtttgttaacggaagcgggcttccgggcctggctatgtacagctcaagcccgatttgggttactggaagagaaagattagctgcttctcactgtgaacgggcccgacaggcccaaggtttcgggcttcagactatggtttgtattccttcagctaatggtgttgttgagctcgggtcaactgagttgatattccagagcgctgatttaatgaacaaggttaaaatcttgtttgattttaatattgatatgggcgcgactacgggctcaggttcgggctcatgtgctattcaggctgagcccgatccttcaaccctttggcttacggatccaccttcctcagttgtggaagtcaaggattcgtcgaatacagttccttcaagtaatagtagtaagcaacttgtgtttggaaatgagaattctgaaaatgttaatcaaaattctcagcaaacacaaggatttttcactagggagttgaatttttccgaatatggatttgatggaagtaatactaggagtggaaatgggaatgtgaattcttcgcgttcttgcaagcctagaaatgcttcaagtgcaaatgggagcttgttttcgggccaatcgcagttcggtcccgggcctgcggaggagaacaagaacaagaacaagaaaaggtcacctgcatcaagaggaagcaatgaagaaggaatgctttcatttgtttcgggtgtgatcttgccaagttcaaacacggggaagtccggtggaggtggcgattcggatcattcagatctcgaggcttcggtggtgaaggaggcggatagtagtagagttgtagaccccgagaagaggccgaggaaacgaggaaggaaaccggctaacgggagagaggagccattgaatcatgtggaggcagagaggcaaaggagggagaaattgaatcaaagattctatgcacttagagctgttgtaccaaatgtgtcaaaaatggataaagcatcacttcttggtgatgcaattgcatttatcaatgagttgaaatcaaaggttcagaattctgactcagataaagatgagttgaggaaccaaattgaatctttaaggaatgaattagccaacaagggatcaaactataccggtcctccaccgccaaatcaagatctcaagattgtagatatggatatcgacgttaaagtcatcggatgggatgctatgattcgtatacaatctaataaaaagaaccatccagccgcgaggttaatggccgctctcatggaattggacttagatgtgcaccatgctagtgtttcagttgtcaacgagttgatgatccaacaagcgacagtgaaaatggggagccggctttacacgcaagagcagcttcggatatcattgacatccagaattgctgaatcgcgatgaagagaaatacagtaaatggaaattattagtgagctctgaataatgttatctttcattgagctattttaagagaatttctcctatagttagatcttgagattaaggctacttaaaagtggaaagttgattgagctttcctcttagttttttgggtatttttcaacttttatatctagtttgttttccacattttctgtacatataatgtgaaaccaatactagatctcaagatctggtttttagttctgtaattagaaataaatatgcagcttcatctttttctgttaaaaaaaaaaaaaaaaaaaaaaaaa

SEQ ID NO: 11 - protein, 658 aa, Nicotiana tabacum
MTDYRIPTMTNIWSNTTSDDNMMEAFLSSDPSSFWAGTNTPTPRSSVSPAPAPVTGIAGDPLKSMPYFNQESLQQRLQTLIDGAREAWTYAIFWQSSVVDFVSPSVLGWGDGYYKGEEDKNKRKTAAFSPDFITEQEHRKKVLRELNSLISGTQTGGENDAVDEEVTDTEWFFLISMTQSFVNGSGLPGLAMYSSSPIWVTGRERLAASHCERARQAQGFGLQTMVCIPSANGVVELGSTELIFQSADLMNKVKILFDFNIDMGATTGSGSGSCAIQAEPDPSTLWLTDPPSSVVEVKDSSNTVPSSNSSKQLVFGNENSENVNQNSQQTQGFFTRELNFSEYGFDGSNTRSGNGNVNSSRSCKPESGEILNFGDSTKRNASSANGSLFSGQSQFGPGPAEENKNKNKKRSPASRGSNEEGMLSFVSGVILPSSNTGKSGGGGDSDHSDLEASVVKEADSSRVVDPEKRPRKRGRKPANGREEPLNHVEAERQRREKLNQRFYALRAVVPNVSKMDKASLLGDAIAFINELKSKVQNSDSDKDELRNQIESLRNELANKGSNYTGPPPPNQDLKIVDMDIDVKVIGWDAMIRIQSNKKNHPAARLMAALMELDLDVHHASVSVVNELMIQQATVKMGSRLYTQEQLRISLTSRIAESR